(19) United States
(12) Patent Application Publication
(10) Pub. No.: US 2013/0040982 A1
(43) Pub. Date: Feb. 14, 2013

(54) ORAL SUSTAINED RELEASE FORMULATION OF HUPERZINE A

(71) Applicants: Michael Friedman, Jerusalem (IL); Amnon HOffman, Jerusalem (IL); Gregory Burshtein, MaAle Adumim (IL)

(72) Inventors: Michael Friedman, Jerusalem (IL); Amnon HOffman, Jerusalem (IL); Gregory Burshtein, MaAle Adumim (IL)

(73) Assignee: Yissum Research Development Company of the Hebrew University of Jerusalem Ltd., Jerusalem (IL)

(21) Appl. No.: 13/641,875

(22) PCT Filed: Apr. 20, 2011
(86) PCT No.: PCT/IB2011/051721

§ 371 Date: Oct. 18, 2012

Related U.S. Application Data
(60) Provisional application No. 61/326,874, filed on Apr. 22, 2010.

(51) Int. Cl. A61K 31/4748; A61P 25/18; A61P 3/00; A61P 25/08; A61P 9/10; A61P 25/28; A61P 29/00

(52) U.S. Cl. 514/295

(57) ABSTRACT

Sustained-release formulations comprising huperzine A are disclosed herein. The formulations are for oral administration, and contain a carrier which comprises native albumin. Unit dosage forms of the formulations, and kits comprising such unit dosage forms are also disclosed herein. Methods utilizing the formulations for treating a medical condition treatable by huperzine A are also disclosed herein, as well as processes for preparing the formulations, and uses of huperzine A and albumin in the manufacture of a medicament.

### FIELD AND BACKGROUND OF THE INVENTION

The present invention, in some embodiments thereof, relates to a pharmaceutical formulation, and more particularly, but not exclusively, to a pharmaceutical formulation comprising huperzine A.

Huperzine A, a sesquiterpene alkaloid, is isolated from the Chinese club moss Huperzia serrata. This herb has been used in China for centuries in the treatment of conditions such as contusions, strains, swelling, and schizophrenia. Huperzine A has been found to improve cognitive deficits in a broad range of animal models and is widely used in China to improve the memory deficits in elderly people and patients with benign senescent forgetfulness, Alzheimer's disease, and vascular dementia [Kelley & Knopman, Neurologist 2008, 14:299-306; Little et al., Expert Opin Investig Drugs 2008, 17:209-215]. In the U.S., huperzine A is marketed in low dosages (up to 50 μg) as a dietary supplement for memory loss and mental impairment [Jiang et al., Curr Med Chem 2003, 10:2231-2252].

Huperzine A has undergone clinical testing for treatment of Alzheimer's disease [Little et al., Expert Opin Investig Drugs 2008, 17:209-215; Xu et al., Zhongguo Yao Li Xue Bao 1999, 20:486-49; Zhang et al., Zhonghua Yi Xue Za Zhi 2002, 82:941-944; Wang et al., J Neural Transm 2009, 116: 457-465]. The results indicate that huperzine A is well-tolerated and is beneficial for Alzheimer's disease patients, particularly when administered at a daily dose of 300-500 μg [Wang et al., J Neural Transm 2009, 116: 457-465; Little et al., Expert Opin Investig Drugs 2008, 17:209-215; Li et al., Cochrane Database Syst Rev 2008, (2): CD005592].

Huperzine A is a reversible, highly specific, potent, and selective acetylcholinesterase (AChE) inhibitor. Huperzine A has better penetration through the blood-brain barrier, higher oral bioavailability, and longer duration of AChE inhibitory action when compared with tacrine, galanthamine, donepezil, and rivastigmine, which have been approved for Alzheimer's disease (AD) in the United States and some European countries [Little et al., Expert Opin Investig Drugs 2008, 17:209-215; Lallement et al., Neurotoxicology 2002, 23:1-5; White et al., Epilepsia 2005, 46(Suppl. 8):220; Schachter et al., Epilepsia 2006, 47(Suppl. 4):319-320]. In addition to its acetylcholinesterase inhibitory effect, huperzine A may have other neuroprotective effects. Huperzine A exhibits an ability to protect cells against hydrogen peroxide, β-amyloid protein (or peptide), glutamate, ischemia and staurosporine-induced cytotoxicity and apoptosis. These protective effects are associated with an ability to attenuate oxidative stress, regulate the expression of apoptotic proteins Bcl-2, Bax, P53, and caspase-3, protect mitochondria, upregulate nerve growth factor and its receptors, and interfere with amyloid precursor protein metabolism [Xu et al., Zhongguo Yao Li Xue Bao 1999, 20: 486-490]. Antagonistic effects of huperzine A towards N-methyl-D-aspartate receptors and potassium currents may also contribute to neuroprotection.

Huperzine A in large oral dosages has been reported to be an effective anticonvulsant against pentylenetetrazol-induced seizures in mice [White et al., Epilepsia 2005, 46(Suppl. 8):220], complex partial seizures in dogs [Schneider et al., Epilepsy Behav 2009, 15:529-534], and in a 6-Hz model of psychomotor seizures [Schachter et al., Epilepsia 2006, 47(Suppl. 4):319-320].

In addition, huperzine A can also be used as a protective agent against organophosphate intoxication, for example, as a prophylactic drug against poisoning by soman and other nerve gases, without causing typical cholinergic side effects. Huperzine A was found to be more effective than pyridostigmine in protecting against soman. The superior protection offered by Huperzine A appears to be associated with the ability of huperzine A (but not pyridostigmine) to selectively inhibit red blood cell acetylcholinesterase activity without inhibiting plasma butyrylcholinesterase, thereby preserving the organophosphate scavenging capacity of butyrylcholinesterase, and to the protection conferred by huperzine A on cerebral acetylcholinesterase [LaBement et al., Neurotoxicology 2002, 23:1-5].

Huperzine A was reported to inhibit pain behavior in a rat formalin pain model [Park et al., Neurosci Lett 2010, 470:6-9]. Similarly, huperzine A induced a dose-dependent antinociception in mice, without altering motor coordination at the maximal effective dose [Galeotti et al., Drug Development Research 2001, 54:19-26].

Pharmacokinetic studies in rodents and dogs indicate that Huperzine A is absorbed rapidly when administered orally, distributed widely in the body, and eliminated at a relatively fast rate.

There are limited data relating to pharmacokinetics of huperzine A in humans. It has been reported that upon oral administration of 0.99 mg to six healthy volunteers, huperzine A was absorbed rapidly, distributed widely in the body and eliminated at a moderate rate. Huperzine A conformed to a one-compartment open model with first absorption and first elimination [Qian et al., Zhongguo Yao Li Xue Bao 1995, 16:396-398]. It has been further reported that Huperzine A exhibited a biphasic profile with rapid distribution followed by a slower elimination rate [Li et al., Eur J Drug Metab Pharmacokinet 2007, 32:183-187]. Peak serum concentration has been reported to be achieved after approximately 1 hour, with reported half-lives in a range of approximately 6-12 hours, and with bioavailability being 99% [Qian et al., Zhongguo Yao Li Xue Bao 1995, 16:396-398; Yuan et al., Chinese Pharmaceutical Journal 2008, 43:1889-1892; Li et al., Eur J Drug Metab Pharmacokinet 2007, 32:183-187].

Animal and clinical safety tests showed that huperzine A has no toxicity other than mild cholinergic side effects, in contrast to the dose-limiting hepatotoxicity exhibited by tacrine.

There are a number of synthetic huperzine A analogs. Huprine X is a fusion product of huperzine A and tacrine. Another synthetic analog is ZT-1, which is a prodrug that is progressively hydrolyzed into huperzine A in the body [Li et al., Biomed Chromatogr 2008, 22:354-360].

Chinese Patent No. 1726911 describes a controlled-release medicament comprising huperzine, a polyacrylic acid cross-linked polymer, and at least one polymer such as polyethylene glycol, polyinyl pyrrolidone, ethylcellulose or β-cyclodextrin.

Chinese Patent Application No. 101081217 describes a slow release huperzine A pill, comprising an inert core, an active layer containing huperzine A which coats the core, and a slow release layer which coats the active layer.

Chinese Patent Application No. 1751683 describes a skeleton-type slow release huperzine A tablet, comprising a hydrophilic gel as skeleton slow release material (20-90 weight percents) and filler (5-75 weight percents).

Chinese Patent Application No. 101485640 describes a controlled release tablet which comprises a tablet core containing huperzine A and a semi-permeable coating membrane coating the tablet core, wherein the coating membrane is provided with a drug release hole. The tablet core comprises a weight ratio of polyethylene glycol to cellulose acetate in a range of from 0.05:1 to 0.25:1.

Chinese Patent Application No. 101485639 describes a controlled release tablet which comprises a double-layer tablet core containing a drug-containing layer and a pushing layer, and a semi-permeable coating membrane coating the tablet core, wherein the coating membrane is provided with a drug release hole. The drug-containing layer contains huperzine A, a suspending agent, osmotic pressure active materials and pharmaceutical excipients. The pushing layer comprises an expanding agent, a permeation enhancer and a binder. The coating comprises polyethylene glycol.

Chinese Patent No. 1682719 describes a slow release enteric soluble coated tablet containing huperzine A. The enteric soluble coating is to facilitate stable, slow and complete release of the drug, which is adversely affected by the low pH in the stomach. The tablets exhibit a sustained release time of 20 hours.

U.S. Pat. No. 4,705,645 describes sustained release tablets comprising an admixture of theophylline and denatured egg albumin, for use in the treatment of asthma. The tablets are prepared by wetting and drying a mixture of theophylline and dried egg albumin, converting the obtained granulates into tablets, and heating the tablets to denature the egg albumin. As further described therein, the process for the manufacture of the tablets requires preparation of granulates of specific size.

U.S. Pat. No. 4,582,719 describes a process for producing a slow release composite having a physiologically active substance encapsulated therein, by contacting the active substance with the protein and heating the mixture with steam to denature the protein, so as to fix and encapsulate the active substance. As further described therein, heating in the absence of water or at a temperature of less than 50° C. is ineffective.

Additional background art includes U.S. Patent Application No. 20070059369, Chinese Patent Application No. 1456151 and Chinese Patent No. 1208054.

### SUMMARY OF THE INVENTION

According to an aspect of some embodiments of the present invention there is provided a sustained-release formulation comprising huperzine A and a carrier, the carrier comprising native albumin, the formulation being for oral administration.

According to some embodiments of the invention, the carrier is a solid carrier.

According to some embodiments of the invention, the albumin is egg albumin.

According to some embodiments of the invention, at least 50 weight percents of the carrier is the native albumin.

According to some embodiments of the invention, a concentration of huperzine A ranges from 0.1 to 10 weight percents of the total weight of the formulation.

According to some embodiments of the invention, the carrier further comprises a polymer.

According to some embodiments of the invention, the polymer is selected from the group consisting of a hydrophobic polymer and a hydrophilic polymer.

According to some embodiments of the invention, the polymer is selected from the group consisting of ethyl cellulose, methyl cellulose, hydroxypropyl cellulose, hydroxypropylmethylcellulose, hydroxyethyl cellulose, hydroxyethylmethylcellulose, carboxymethyl cellulose, poly(methacrylic acid-co-methyl methacrylate), poly(methacrylic acid-co-ethyl acrylate), poly(ethylene oxide), a poloxamer, a polyacrylamide, a polysaccharide, and a protein.

According to some embodiments of the invention, the polymer is selected from the group consisting of ethyl cellulose, hydroxypropylmethylcellulose, carboxymethyl cellulose, hydroxypropyl cellulose, poly(ethylene oxide), poly(methacrylic acid-co-methyl methacrylate) and poly(methacrylic acid-co-ethyl acrylate).

According to some embodiments of the invention, a concentration of the polymer ranges from 5 to 60 weight percents.

According to some embodiments of the invention, the carrier further comprises an additional component selected from the group consisting of a saccharide, and a fatty substance.

According to some embodiments of the invention, the saccharide is selected from the group consisting of a monosaccharide and a disaccharide.

According to some embodiments of the invention, the saccharide is lactose.

According to some embodiments of the invention, a concentration of lactose ranges from 10 to 70 weight percents of the total weight of the carrier.

According to some embodiments of the invention, the fatty substance comprises a compound selected from the group consisting of a fatty alcohol, a fatty acid, and a fatty acid ester.

According to some embodiments of the invention, the formulation comprises huperzine A in an amount selected from the group consisting of 0.4 weight percent, 0.5 weight percent, and 1 weight percent of the total weight of the formulation, with the balance being native egg albumin.

According to some embodiments of the invention, the formulation comprises huperzine A, native egg albumin and hydroxypropylmethylcellulose.

According to some embodiments of the invention, the formulation comprises 1 weight percent huperzine A, 79.5 weight percents native egg albumin, and 19.5 weight percents hydroxypropylmethylcellulose.

According to some embodiments of the invention, the formulation comprises 0.4 weight percent huperzine A, 69.6 weight percents native egg albumin, and 30 weight percents hydroxypropylmethylcellulose.

According to some embodiments of the invention, the formulation comprises huperzine A, native egg albumin and ethyl cellulose.

According to some embodiments of the invention, the formulation comprises 1 weight percent huperzine A, 59.5 weight percents native egg albumin, and 39.5 weight percents ethyl cellulose.

According to some embodiments of the invention, the formulation comprises huperzine A, native egg albumin and carboxymethyl cellulose.

According to some embodiments of the invention, the formulation comprises 0.4 weight percent huperzine A, 69.6 weight percents native egg albumin, and 30 weight percents carboxymethyl cellulose.

According to some embodiments of the invention, the formulation comprises huperzine A, native egg albumin and poly(methacrylic acid-co-methyl methacrylate).

According to some embodiments of the invention, the formulation comprises 0.4 weight percent huperzine A, and poly(methacrylic acid-co-methyl methacrylate) in an amount selected from the group consisting of 10 weight percents, 20 weight percents, 30 weight percents and 40 weight percents, with the balance being native egg albumin.

According to some embodiments of the invention, the formulation comprises huperzine A, native egg albumin and poly(ethylene oxide).

According to some embodiments of the invention, the formulation comprises 0.4 weight percent huperzine A, 69.6 weight percents native egg albumin, and 30 weight percents poly(ethylene oxide).

According to some embodiments of the invention, the formulation comprises huperzine A, native egg albumin and hydroxypropyl cellulose.

According to some embodiments of the invention, the formulation comprises 0.4 weight percent huperzine A, 69.6 weight percents native egg albumin, and 30 weight percents hydroxypropyl cellulose.

According to some embodiments of the invention, the formulation is characterized by a release of 50% of the huperzine A upon incubation in 0.2 M phosphate buffer at a pH of 6.8 and a temperature of 37° C. for a time period in a range of from 1 to 10 hours.

According to some embodiments of the invention, the formulation is characterized by a release of from 10% to 40% of the huperzine A upon incubation in 0.2 M phosphate buffer at a pH of 6.8 and a temperature of 37° C. for 15 minutes.

According to some embodiments of the invention, the formulation is characterized by a release of from 30% to 50% of the huperzine A upon incubation for 30 minutes at 37° C. in U.S. Pharmacopeia simulated gastric fluid.

According to some embodiments of the invention, the formulation is characterized by an ability, upon oral administration of the formulation to a human subject, to maintain a plasma concentration of huperzine A which is at least 30% of the maximal plasma concentration, for at least 24 hours.

According to some embodiments of the invention, the formulation is a unit dosage form formulation.

According to some embodiments of the invention, the formulation is characterized by an ability, upon oral administration of the unit dosage form to a human subject, to maintain a plasma concentration of at least 0.75 ng/ml huperzine A for at least 24 hours.

According to some embodiments of the invention, the formulation is in a tablet form.

According to some embodiments of the invention, the unit dosage form formulation (e.g., a tablet) comprises huperzine A in an amount that ranges from 20 ng to 10 mg.

According to some embodiments of the invention, the formulation is identified for use in treating a medical condition treatable by huperzine A.

According to some embodiments of the invention, the formulation (or unit dosage form formulation) is for being administered once per day.

According to some embodiments of the invention, the formulation is packaged in a packaging material and identified, in or on the packaging material, for use in the treatment of a medical condition treatable by huperzine A.

According to as aspect of some embodiments of the present invention there is provided a kit comprising a plurality of the unit dosage form formulations as described herein, and instructions for using the unit dosage form for treating a medical condition treatable by huperzine A.

According to as aspect of some embodiments of the present invention there is provided a method of treating a medical condition treatable by huperzine A, the method comprising orally administering the formulation or formulation unit dosage form of as described herein to a subject in need thereof, thereby treating the medical condition.

According to some embodiments of the invention, the administering is effected once per day.

According to as aspect of some embodiments of the present invention there is provided a use of native albumin and huperzine A in the manufacture of a medicament for treating a medical condition treatable by huperzine A.

According to some embodiments of the invention, the medicament is for being administered orally.

According to some embodiments of the invention, the medicament is for being administered once per day.

According to some embodiments of the invention, the medical condition is associated with an activity of a protein selected from the group consisting of an acetylcholine esterase and an N-methyl-D-aspartate receptor.

According to some embodiments of the invention, the medical condition is selected from the group consisting of Alzheimer's disease, memory loss, vascular dementia, schizophrenia, inflammation, organophosphate intoxication, epilepsy, ischemia, and pain.

According to as aspect of some embodiments of the present invention there is provided a process of preparing the formulation of any of claims 1 to 39, the process comprising blending huperzine A and native albumin so as to form a homogeneous mixture.

According to some embodiments of the invention, the process further comprises compressing the mixture so as to obtain a tablet form of the formulation.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a bar graph showing the permeability coefficient (Papp) for huperzine A (HupA), antipyrine and mannitol, as determined in a Caco-2 cell model;

FIG. 2 is a bar graph showing the permeability coefficient (Papp) for huperzine A (HupA) in the duodenum, jejunum and colon, and for antipyrine, as determined in intestinal tissue in an ex-vivo Ussing chamber model;

FIG. 3 is a graph showing the plasma concentration of huperzine A in rats, as a function of time following administration of 0.5 mg/kg huperzine A by intravenous infusion (IV) or by oral gavage (PO);

FIG. 4 is a graph showing the plasma concentration of huperzine A in rats, as a function of time following administration of 0.5 mg/kg huperzine A by intraperitoneal (IP) injection or by oral gavage;

FIG. 5 is a graph showing the plasma concentration of huperzine A in rats, as a function of time following administration of 0.5 mg/kg huperzine A by cecal infusion (Cecum inf.), duodenal infusion (Duodenal inf.), or by oral gavage (PO);

FIG. 6 is a graph showing the plasma concentration of huperzine A in rats, as a function of time following oral administration of 0.5 mg/kg huperzine A in the form of a solution (PO—solution), a controlled release tablet comprising a matrix of egg albumin and lactose (CR tablet (albumin/lactose)), or a controlled release tablet comprising a matrix of egg albumin (CR tablet (albumin));

FIG. 7 is a bar graph showing the percentage of huperzine A which remained following exposure to a temperature of 120° C., 150° C. or 190° C., for 30, 60 or 90 minutes;

FIG. 8 is a graph showing the percentage of huperzine A released from tablets containing egg albumin denatured by ethanol or heat, or undenatured egg albumin, as a function of time of dissolution of the tablet in phosphate buffer (pH 6.8);

FIG. 9 is a graph showing the percentage of huperzine A released from a 100 mg tablet with a 5 mm diameter, a 100 mg tablet with a 7 mm diameter, and a 200 mg tablet with a 7 mm diameter, as a function of time of dissolution of the tablet in phosphate buffer (pH 6.8);

FIG. 10 is a graph showing the percentage of huperzine A released from tablets containing a matrix of egg albumin, 80% egg albumin and 20% lactose, or 50% egg albumin and 50% lactose, as a function of time of dissolution in phosphate buffer (pH 6.8);

FIG. 11 is a graph showing the percentage of huperzine A released from tablets containing a matrix of egg albumin, or 80% egg albumin and 20% hydroxypropylmethylcellulose (K100LV), as a function of time of dissolution in phosphate buffer (pH 6.8);

FIG. 12 is a graph showing the percentage of huperzine A released from tablets containing a matrix of egg albumin, ethyl cellulose, or 60% egg albumin and 40% ethyl cellulose (EC), as a function of time of dissolution in phosphate buffer (pH 6.8);

FIG. 13 is a graph showing the percentage of huperzine A released from tablets containing a matrix of 70% egg albumin and 30% carboxymethyl cellulose, as a function of time of dissolution in phosphate buffer (pH 6.8);

FIG. 14 is a graph showing the percentage of huperzine A released from tablets containing a matrix of egg albumin (diamonds), or egg albumin with 10% (triangles), 20% (crosses), 30% (circles) or 40% Eudragit® L 100, as a function of time of dissolution in phosphate buffer (pH 6.8);

FIG. 15 is a graph showing the percentage of huperzine A released from tablets containing 0.4% huperzine A and 99.6% native egg albumin, or 0.4% huperzine A, 69.6% native egg albumin, and 30% of hydroxypropylmethylcellulose (HPMC K100LV CR) or poly(ethylene oxide) (Polyox WSR-303), as a function of time of dissolution in phosphate buffer (pH 6.8);

FIG. 16 is a graph showing the percentage of huperzine A released from tablets containing 0.4% huperzine A and 99.6% native egg albumin, or 0.4% huperzine A, 69.6% native egg albumin and 30% hydroxypropyl cellulose (Klucel® HF), as a function of time of dissolution in phosphate buffer (pH 6.8);

FIG. 17 is graph showing the percentage of huperzine A released from tablets containing 0.4% huperzine A and 99.6% egg albumin, as a function of time of dissolution in phosphate buffer (pH 6.8) or simulated biological fluids;

FIG. 18 is a graph showing the percentage of huperzine A released from tablets formed from an instant release (IR) formulation, and exemplary sustained release formulations containing a matrix of egg albumin (CR-1) or 80% egg albumin and 20% Eudragit® L 100 (CR-2), as a function of time of dissolution in phosphate buffer (pH 6.8); and

FIG. 19 is a graph showing the plasma concentration of huperzine A in humans, as a function of time following oral administration of 0.4 mg huperzine A in the form of an instant release (IR) formulation solution, or a controlled release (CR) formulation comprising a matrix of egg albumin.

### DESCRIPTION OF SPECIFIC EMBODIMENTS OF THE INVENTION

The present invention, in some embodiments thereof, relates to a pharmaceutical formulation, and more particularly, but not exclusively, to a pharmaceutical formulation comprising huperzine A.

The principles and operation of the present invention may be better understood with reference to the figures and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Huperzine A has shown considerable promise as a therapeutically active agent for treating a variety of medical conditions. However, the limited available pharmacokinetic data in humans suggests that Huperzine A would need to be administered multiple times per day in order to achieve a consistent therapeutically effective plasma concentration of the drug.

In an attempt to improve treatment of medical conditions which are treatable by Huperzine A, the present inventors have studied in detail the absorption and pharmacokinetics of huperzine A using in vitro and in vivo models. Based on these studies, the inventors have designed inexpensive and simple-to-produce sustained-release formulations, which release huperzine A at a rate which allows for more stable levels of huperzine A in vivo, and therefore more effective treatment.

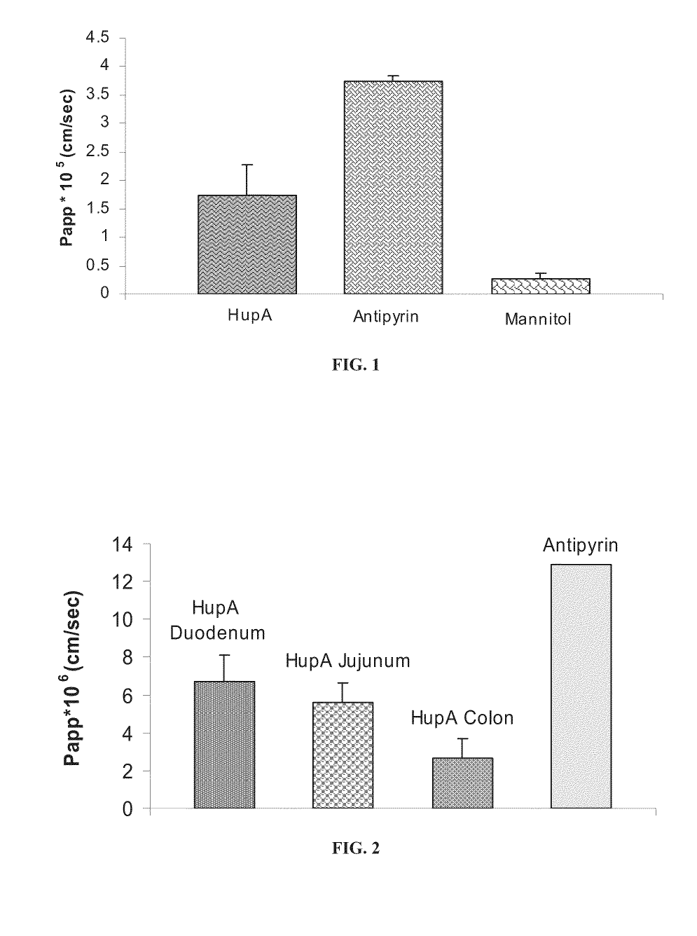

Referring now to the drawings, FIGS. 1 and 2 show the permeability of intestinal cells to huperzine A in an in vitro cell line model (FIG. 1) and in ex vivo intestinal tissue (FIG. 2). FIG. 2 shows that huperzine A is more efficiently absorbed in the duodenum and jejunum than in the colon.

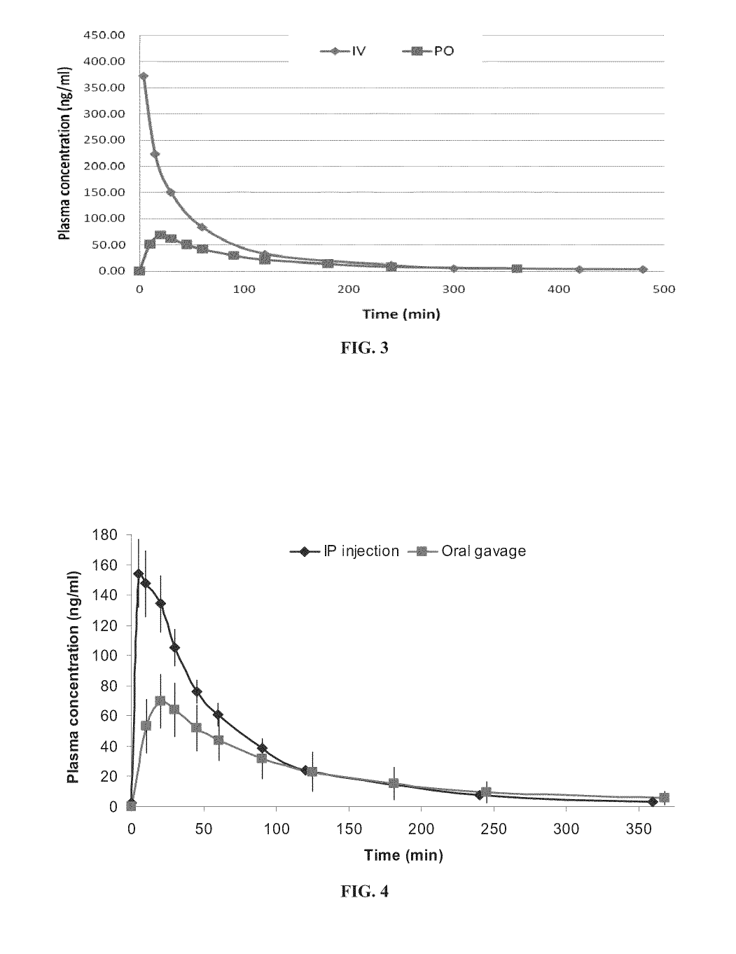

FIGS. 3 and 4 show the effect of oral, intravenous and intraperitoneal administration of huperzine A on plasma concentrations of huperzine A in an in vivo rat model. These results indicate that the oral bioavailability is approximately 50%.

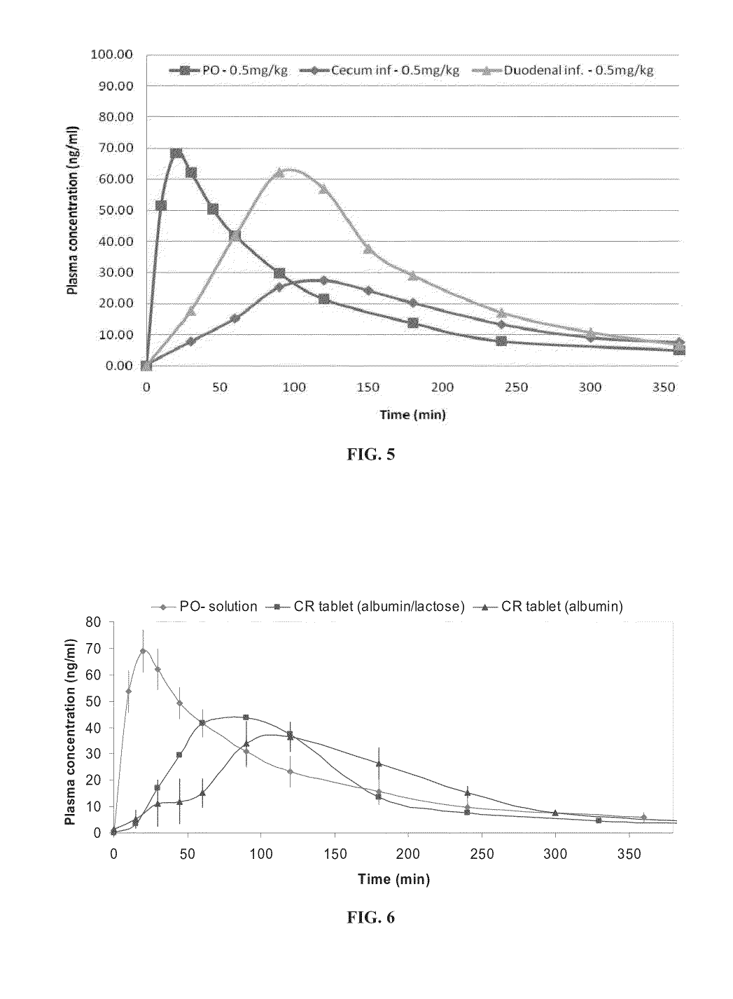

FIG. 5 shows that the bioavailability of huperzine A for cecal infusion is lower than the bioavailability for oral administration and duodenal infusion. These results confirm that huperzine A is less efficiently absorbed in the colon.

The present inventors have surprisingly uncovered that formulating huperzine A in a carrier that comprises native albumin provides for a desired release profile of the drug, without compromising the activity of the huperzine A.

FIG. 6 shows that a native egg-albumin matrix results in more gradual uptake of huperzine A into the blood, without having any significantly adverse impact on bioavailability of the drug.

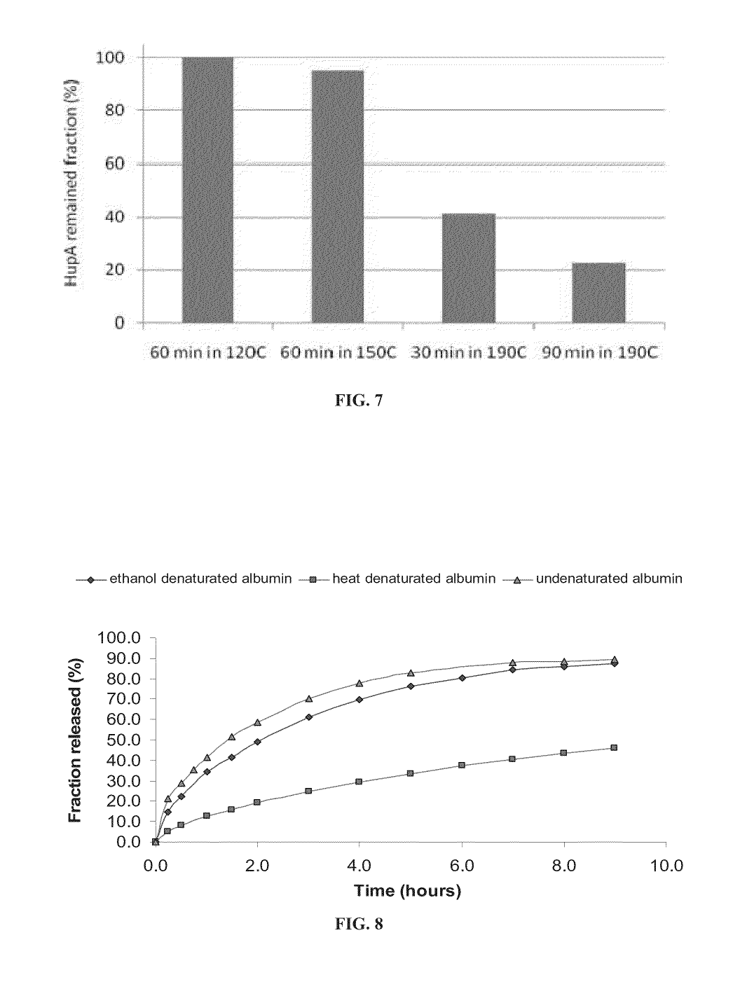

FIG. 7 shows that huperzine A can be degraded by heat treatment.

FIG. 8 shows the effect of denaturation treatments on the release of huperzine A from an exemplary egg albumin-based formulation.

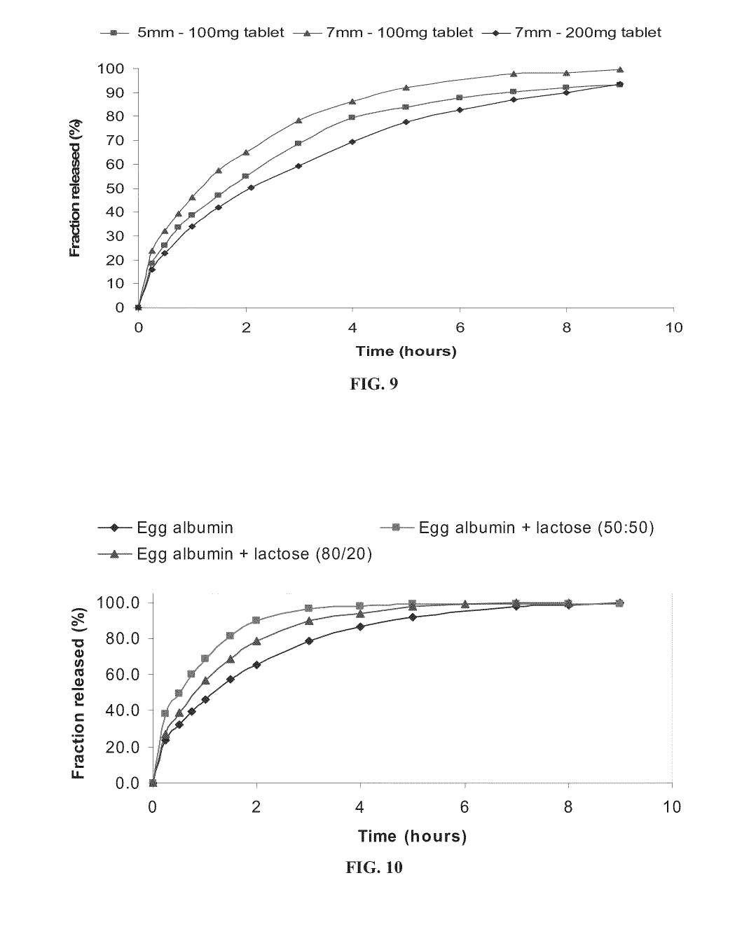

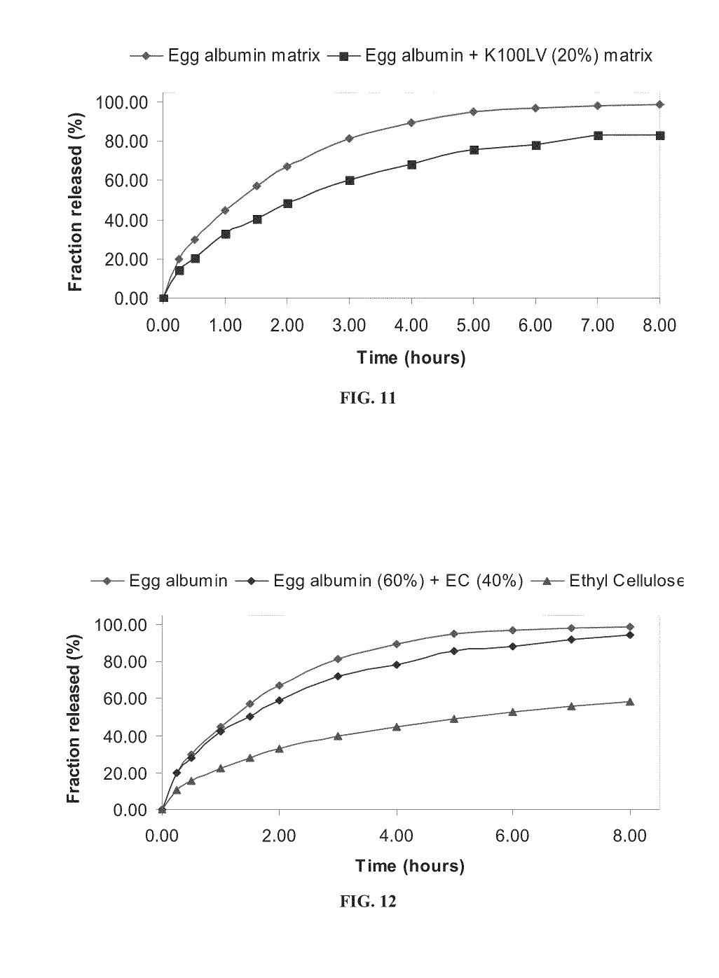

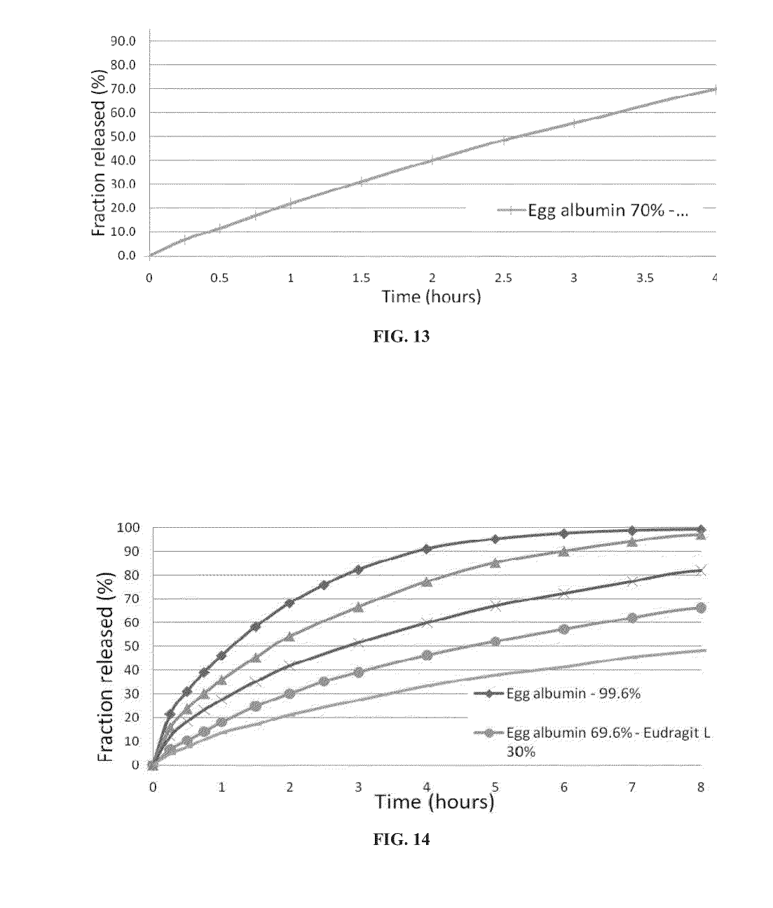

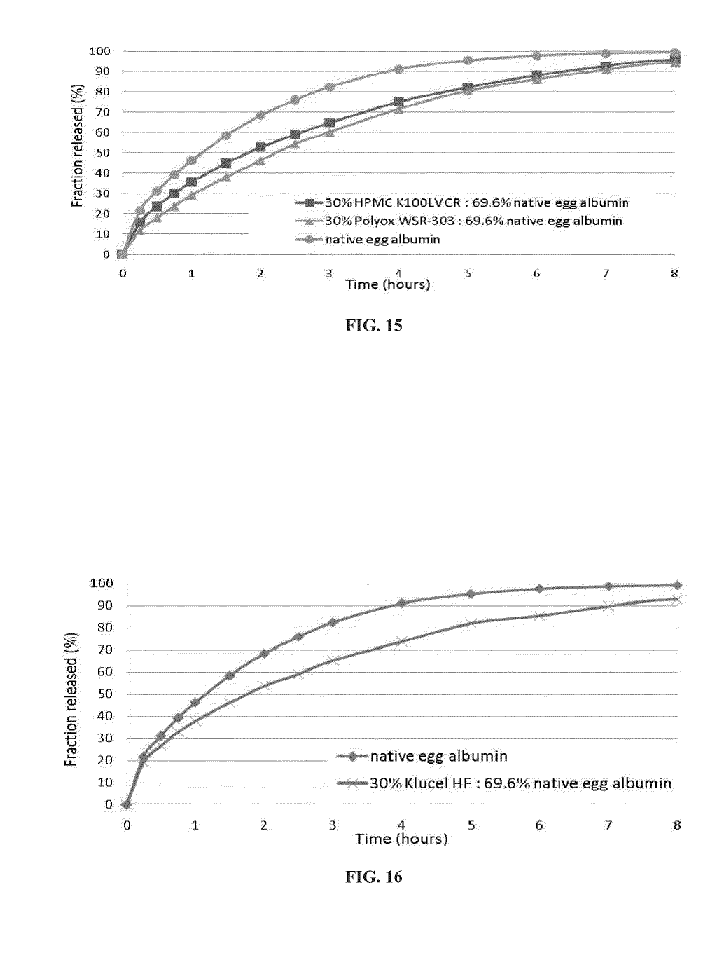

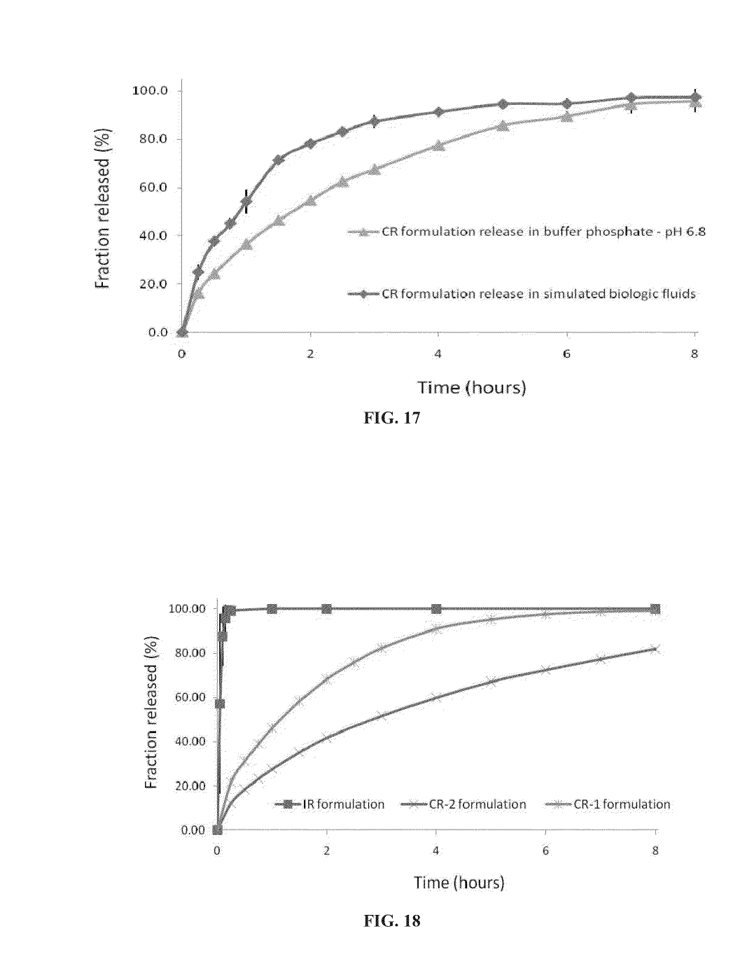

FIGS. 9-16 show that the release profile of huperzine A from native egg albumin-based formulations can be modulated by the size and shape of a tablet formulation (FIG. 9), addition of lactose (FIG. 10), hydroxypropylmethylcellulose (FIGS. 11 and 15), ethyl cellulose (FIG. 12), carboxymethyl cellulose (FIG. 13), Eudragit® L 100 (FIG. 14), poly(ethylene oxide) (FIG. 15), and hydroxypropyl cellulose (FIG. 16). FIG. 17 shows the effect of the different conditions in the stomach and intestines on release of huperzine A from an exemplary native egg albumin-based formulation. FIG. 18 shows release profiles of exemplary native egg albumin-based formulations.

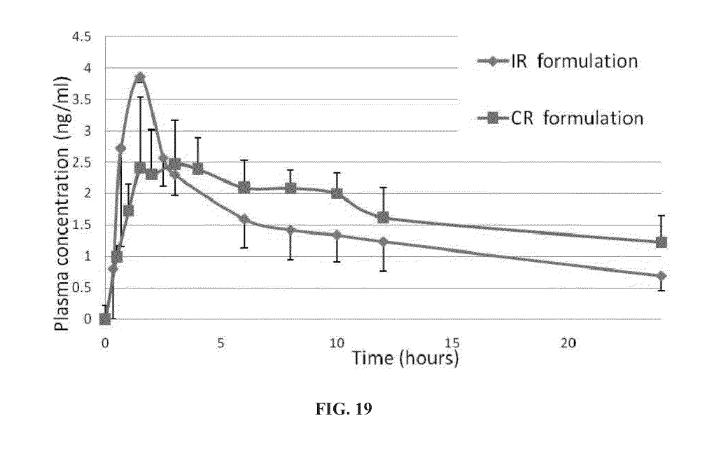

FIG. 19 shows that plasma concentrations in humans are relatively stable for at least about 24 hours following oral administration of an exemplary albumin-based formulation according to an embodiment of the invention.

As demonstrated in the Examples section that follows, the present inventors have shown that oral administration of native albumin-based sustained-release formulations according to embodiments of the present invention can provide a relatively stable plasma concentration for 24 hours, characterized in both a reduced peak concentration (Cₘₐₓ) and in a higher minimal concentration (at the end of the 24 hours). The relatively stable plasma concentration is particularly desirable, for example, in a medicament for being administered once per day. As further demonstrated therein, the huperzine A is efficiently absorbed upon oral administration of such native albumin-based formulations.

Hence, according to an aspect of embodiments of the invention, there is provided a sustained-release formulation for oral administration. The formulation comprises huperzine A and a carrier, and the carrier comprises native albumin.

As used herein, the term “sustained-release” refers to a formulation which upon administration (e.g., oral administration) releases a substantial amount (e.g., at least 30 weight percents, at least 40 weight percents, at least 50 weight percents, at least 60 weight percents, at least 70 weight percents or at least 80 weight percents) of the drug contained in the formulation (e.g., huperzine A) continuously during the course of a prolonged time period. A “prolonged time period” means for at least 2 hours, for at least 4 hours, and optionally for at least 6 hours. In the context of the embodiments described herein, the term “sustained-release” (in accordance with the aforementioned definition) is to be interpreted as meaning “slow release”.

Thus, a sustained-release formulation is to be distinguished from both an immediate release formulation, in which at least most of the drug is released immediately or shortly upon administration but does not last for a long time (burst release), as well a delayed-release formulation, in which substantial drug release begins only a considerable time after administration, but does not necessarily last for a long time thereafter.

A formulation may be both a sustained-release formulation and a delayed-release formulation, for example, if drug release begins, e.g., six hours after administration and continuous for, e.g., six hours thereafter (i.e., until 12 hours after administration). However, in the context of the present invention, such a release profile may be undesirable. For example, the formulation may cause a considerable portion of huperzine A to be released in the colon, where absorption is less efficient.

Hence, according to some embodiments, the sustained-release formulation is not characterized by a delayed-release.

A sustained-release formulation, according to some embodiments of the present invention, can be defined also as a formulation that when subjected to physiological conditions, releases 50% of the drug (e.g., huperzine A) during no less than 30 minutes, preferably during no less than 1 hour.

A sustained-release formulation for oral administration is typically designed such that an effective plasma concentration, as defined herein, of an active agent (a drug, e.g., huperzine A), is maintained for a prolonged time period, and thus enables a reduced number of oral administrations during the treatment period. In some embodiments, an effective plasma concentration of the drug is maintained at least for 24 hours, so as to allow administration once per day.

As used herein, an “effective plasma concentration” is defined as at least 10%, at least 20%, at least 30 5, at least 40% or at least 50% of the maximal plasma concentration.

Herein, the term “huperzine A” encompasses huperzine A per se, as presented by the chemical structure below, including stereoisomers and isomorphs thereof, as well as huperzine A in the form of a prodrug and/or derivative of huperzine A, as a pharmaceutically acceptable salt thereof, and in a form of a solvate or hydrate of any of the forgoing.

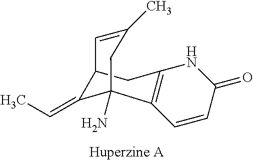 [SMILES: C/C=C1\C2C=C(C)CC1(N)c1ccc(=O)[nH]c1C2]

Herein, a “prodrug” of huperzine A refers to a compound which is hydrolyzed into huperzine A under physiological conditions (e.g., in a subject's body).

Suitable prodrugs and derivatives of huperzine A are described for example, in Li et al. [Biomed Chromatogr 2008, 22:354-360].

ZT-1 is an example of a prodrug of huperzine A which may be used in some embodiments of the present invention.

A derivative of huperzine A may be, for example, a fusion product of huperzine A with another compound. For example, Huprine X is a fusion product of huperzine A and tacrine.

The huperzine A (or prodrug and/or derivative thereof) can be in a form of a pharmaceutically acceptable salt thereof.

As used herein, the phrase “pharmaceutically acceptable salt” refers to a charged species of the parent compound and its counter-ion, which is typically used to modify the solubility characteristics of the parent compound and/or to reduce any significant irritation to an organism by the parent compound, while not abrogating the biological activity and properties of the administered compound.

In the context of some of the present embodiments, a pharmaceutically acceptable salt of the huperzine A (or prodrug and/or derivative thereof) may optionally be an acid addition salt comprising at least one basic (e.g., amine) group of the compound which is in a positively charged form (e.g., an ammonium ion), in combination with at least one counter-ion, derived from the selected acid, that forms a pharmaceutically acceptable salt.

The acid addition salts of the huperzine A (or prodrug and/or derivative thereof) described herein may therefore be complexes formed between one or more amino groups of the drug and one or more equivalents of an acid.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt.

Each of these acid addition salts can be either a mono-addition salt or a poly-addition salt, as these terms are defined herein.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 (for example, 2:1), such that the addition salt includes two or more molar equivalents of the counter-ion per one molar equivalent of the compound.

Further, each of the compounds described herein, including the salts thereof, can be in a form of a solvate or a hydrate thereof.

The term “solvate” refers to a complex of variable stoichiometry (e.g., di-, tri-, tetra-, penta-, hexa-, and so on), which is formed by a solute (the heterocyclic compounds described herein) and a solvent, whereby the solvent does not interfere with the biological activity of the solute.

The term “hydrate” refers to a solvate, as defined hereinabove, where the solvent is water.

The present embodiments further encompass any stereoisomers (enantiomers and diastereomers) of the huperzine A or prodrug and/or derivative thereof described herein, as well as any isomorph thereof.

In some embodiments, the active agent is huperzine per se (not a prodrug or derivative thereof) or a salt thereof, as described herein.

As used herein and in the art, the term “albumin” refers to any protein that is water-soluble, which is moderately soluble in concentrated aqueous salt solutions, and which undergoes heat denaturation. Examples of albumin include serum albumins (e.g., human serum albumin, bovine serum albumin), egg albumin (e.g., ovalbumin) and albumin derived from seeds (e.g., soybean albumin).

According to exemplary embodiments, the albumin is egg albumin

The egg albumin may be obtained from a commercial source or be synthetically prepared (e.g., by expression of recombinant proteins).

Optionally, the egg albumin consists substantially of ovalbumin (e.g., chicken ovalbumin).

Alternatively, the egg albumin comprises ovalbumin (e.g., chicken ovalbumin) along with additional egg proteins. Optionally, at least 60 weight percents of the egg albumin is ovalbumin. Optionally at least 70 weight percents, optionally at least 80 weight percents, optionally at least 90 weight percents, and optionally at least 95 weight percents of the egg albumin is ovalbumin.

The ovalbumin may be a naturally occurring ovalbumin, i.e., an ovalbumin expressed by an organism and/or in an egg of the organism (e.g. chicken ovalbumin), and/or a protein homologous to a naturally occurring ovalbumin. The ovalbumin may be at least 80% homologous, optionally at least 90% homologous, optionally at least 95% homologous, optionally at least 98% homologous, and optionally at least 99% homologous to a naturally occurring ovalbumin (e.g., chicken ovalbumin).

It is to be appreciated that albumins may be obtained at a far lower cost than synthetic polymers which are commonly used to prepare sustained-release formulations.

As used herein, the term “native albumin” refers to albumin which has not been denatured, i.e., albumin which substantially retains its native secondary and tertiary structure.

The albumin may optionally be covalently modified, for example, by cross-linking the albumin with a suitable cross-linking agent.

However, according to exemplary embodiments, the albumin is not covalently modified.

Denaturation of proteins tends to cause the proteins to be less soluble in water and/or to aggregate, thereby leading to hardening of a protein-containing substance.

This phenomenon is utilized in U.S. Pat. Nos. 4,705,645 and 4,582,719, which describe specific processes for forming a tablet, in which a mixture of a protein and a drug is subjected to denaturation (e.g., by heating), so as to encapsulate the drug in a solid matrix of denatured protein.

In view of the above, denaturing the protein prior to mixing the protein with the drug would not be expected to provide an advantage, as the reduced solubility and/or aggregation caused by the denaturation would likely make the protein more difficult to work with (e.g., harder to mix with the drug), without serving the purpose of encapsulating the drug in a solid matrix.

However, as described in the Examples section below, denaturation of a protein (e.g., albumin) in a formulation by heat treatment may cause degradation of the huperzine A in the formulation, whereas denaturation of a protein in a formulation by chemical means (e.g., by exposure to ethanol) is relatively complicated, in view of the need to avoid damaging the other components of the formulation and/or contaminating the formulation. Furthermore, as described in the Examples section below, denaturation of albumin by chemical means has little effect on the release profile of the formulations.

However, as further described in the Examples section below, it was surprisingly uncovered that native albumin facilitates sustained-release of huperzine A, thereby overcoming the difficulties described hereinabove.

Thus, the use of native albumin is advantageous as it surprisingly provides an effective sustained-release formulation, while avoiding the degradation of huperzine A (e.g., by heat treatment) and minimizing the number of synthetic steps required to prepare the formulation (e.g., by avoiding the complexity of chemical denaturation and/or any other process for treating the albumin).

As used herein, the term “carrier” refers to a substance in which the huperzine A is embedded. Thus, the term “carrier” does not include, for example, capsule shells which encapsulate the huperzine A, or coatings (e.g., enteric coatings) or other layers (e.g., inert layers) in a dosage form which do not include huperzine A therein.

Optionally, the carrier is a solid carrier, which is also referred to herein as a “matrix”. The matrix optionally provides a sustained release of the huperzine A embedded therein by dissolving in the gastrointestinal tract over the course of at least 4 hours, and optionally at least 6 hours.

The rate of dissolution of the matrix will usually affect the rate of huperzine A release. For example, in general, when a matrix has completely dissolved, essentially all of the huperzine A will have been released. However, the rate of release may be affected by other factors (e.g., rate of diffusion of huperzine A through the formulation) in addition to, or instead of, the dissolution of the matrix.

According to optional embodiments, the carrier primarily consists of native albumin, i.e., at least 50 weight percents of the carrier is native albumin. Optionally, at least 60 weight percents of the carrier is native albumin, optionally at least 70 weight percents, optionally at least 80 weight percents, and optionally at least 90 weight percents of the carrier is native albumin. Optionally, the carrier essentially consists of native albumin.

The carrier may optionally include components in addition to the native albumin. Preferably, the components of the carrier are selected such that they do not cause significant irritation to an organism and do not abrogate the biological activity and properties of the huperzine A.

The additional components may be for increasing or decreasing a rate of huperzine A release from the formulation (e.g., as described herein), and/or for facilitating manufacture of the formulation (e.g., enhancing a batch-to-batch uniformity of the formulation), improving palatability of the formulation, or any other purpose.

According to optional embodiments, the carrier further comprises one or more polymer(s) in addition to the native albumin. The one or more polymer(s) may optionally comprise a hydrophilic polymer (e.g., water-soluble polymer) and/or a hydrophobic polymer.

Examples of polymers which may optionally be included in the carrier in addition to native albumin include, without limitation, polymers such as cellulose derivatives (e.g., ethyl cellulose, methyl cellulose, hydroxypropyl cellulose, hydroxypropylmethylcellulose, hydroxyethyl cellulose, hydroxyethylmethylcellulose, carboxymethyl cellulose); polyacrylamides; (meth)acrylic acid-(meth)acrylate copolymers such as poly(methacrylic acid-co-methyl methacrylate) and poly(methacrylic acid-co-ethyl acrylate) (e.g., Eudragit® L copolymers); poly(ethylene oxide) and copolymers thereof, such as poloxamers (poly(ethylene oxide-co-propylene oxide)); polysaccharides (e.g., alginate, arabinogalactan, chitosan); and proteins (i.e., proteins other than native albumin).

As demonstrated in the Examples section below, a release profile of the sustained-release formulation may be manipulated according to the type and concentration of a polymer used in combination with the native albumin in the carrier.

Thus, for example, the rate of release of huperzine A from the formulation may optionally be decreased by adding a suitable polymer (e.g., a polymer described hereinabove) to the carrier at a suitable concentration (the effect of the additional polymer is typically concentration dependent).

Exemplary polymers which when added to carrier may decrease the release rate of huperzine A include, but are not limited to, ethyl cellulose, hydroxypropylmethyl cellulose (HPMC), carboxymethyl cellulose, hydroxypropyl cellulose, poly(ethylene oxide), and polymers such as used in Eudragit® L (i.e., poly(methacrylic acid-co-methyl methacrylate) and poly(methacrylic acid-co-ethyl acrylate)), particularly poly(methacrylic acid-co-methyl methacrylate).

Alternatively or additionally, as exemplified below, the release of huperzine A can be made to be an approximately zero-order release (i.e., relatively constant over time) by including a polymer such as carboxymethyl cellulose at a suitable concentration, such as from 10 to 50 weight percents of the carrier.

In some embodiments, the carrier comprises ethyl cellulose. Optionally, the carrier consists essentially of albumin and ethyl cellulose. A concentration of ethyl cellulose optionally ranges from 20 to 60 weight percents of the total weight of the carrier, and optionally from 30 to 40 weight percents. In exemplary embodiments, a concentration of ethyl cellulose is about 40 weight percents of the carrier.

In exemplary embodiments, the formulation comprises huperzine A, native egg albumin, and ethyl cellulose. In some embodiments, the formulation comprises about 1 weight percent huperzine A, about 59.5 weight percents native egg albumin, and about 39.5 weight percents ethyl cellulose.

In some embodiments, the carrier comprises hydroxypropylmethylcellulose. Optionally, the carrier consists essentially of albumin and hydroxypropylmethylcellulose. A concentration of hydroxypropylmethylcellulose optionally ranges from 10 to 60 weight percents of the total weight of the carrier, and optionally from 15 to 50 weight percents.

In some embodiments, a concentration of hydroxypropylmethylcellulose optionally ranges from 15 to 25 weight percents of the total weight of the carrier. In exemplary embodiments, the concentration is about 20 weight percents.

In alternative embodiments, a concentration of hydroxypropylmethylcellulose optionally ranges from 30 to 50 weight percents of the total weight of the carrier. In exemplary embodiments, the concentration is about 40 weight percents.

In exemplary embodiments, the formulation comprises huperzine A, native egg albumin, and hydroxypropylmethylcellulose. In some embodiments, the formulation comprises about 1 weight percent huperzine A, about 79.5 weight percents native egg albumin, and about 19.5 weight percents hydroxypropylmethylcellulose. In alternative embodiments, the formulation comprises about 0.4 weight percent huperzine A, about 69.6 weight percents native egg albumin, and about 30 weight percents hydroxypropylmethylcellulose.

In some embodiments, the carrier comprises carboxymethyl cellulose. Optionally, the carrier consists essentially of albumin and carboxymethyl cellulose. A concentration of carboxymethyl cellulose optionally ranges from 10 to 50 weight percents of the total weight of the carrier, and optionally from 20 to 40 weight percents. In exemplary embodiments, a concentration of carboxymethyl cellulose is about 30 weight percents of the carrier. As described herein, such concentrations of carboxymethyl cellulose in the carrier may decrease the release rate of huperzine A and cause the formulation to exhibit an approximately a zero-order release profile.

In exemplary embodiments, the formulation comprises huperzine A, native egg albumin, and carboxymethyl cellulose. In some embodiments, the formulation comprises about 0.4 weight percent huperzine A, about 69.6 weight percents native egg albumin, and about 30 weight percents carboxymethyl cellulose.

In some embodiments, the carrier comprises poly(ethylene oxide). Optionally, the carrier consists essentially of albumin and poly(ethylene oxide). A concentration of poly(ethylene oxide) optionally ranges from 10 to 50 weight percents of the total weight of the carrier, and optionally from 20 to 40 weight percents. In exemplary embodiments, a concentration of poly(ethylene oxide) is about 30 weight percents of the carrier.

In exemplary embodiments, the formulation comprises huperzine A, native egg albumin, and poly(ethylene oxide). In some embodiments, the formulation comprises about 0.4 weight percent huperzine A, about 69.6 weight percents native egg albumin, and about 30 weight percents poly(ethylene oxide).

Optionally, the poly(ethylene oxide) is characterized by a molecular weight of at approximately 7,000,000 Da (e.g., from 4,000,000 to 10,000,000). Exemplary poly(ethylene oxide) is available as Polyox WSR-303.

In some embodiments, the carrier comprises hydroxypropyl cellulose. Optionally, the carrier consists essentially of albumin and hydroxypropyl cellulose. A concentration of hydroxypropyl cellulose optionally ranges from 10 to 50 weight percents of the total weight of the carrier, and optionally from 20 to 40 weight percents. In exemplary embodiments, a concentration of hydroxypropyl cellulose is about 30 weight percents of the carrier.

In exemplary embodiments, the formulation comprises huperzine A, native egg albumin, and hydroxypropyl cellulose. In some embodiments, the formulation comprises about 0.4 weight percent huperzine A, about 69.6 weight percents native egg albumin, and about 30 weight percents hydroxypropyl cellulose.

Optionally, the hydroxypropyl cellulose is characterized by a molecular weight of approximately 1,000,000 Da (e.g., from 500,000 to 1,500,000). Exemplary hydroxypropyl cellulose is available as Klucel® HF.

In some embodiments, the carrier comprises a polymer such as poly(methacrylic acid-co-methyl methacrylate) and/or poly(methacrylic acid-co-ethyl acrylate). In exemplary embodiments, the polymer is poly(methacrylic acid-co-methyl methacrylate). Optionally, the carrier consists essentially of albumin and the aforementioned polymer. The aforementioned polymer is optionally a polymer available as Eudragit® L (e.g., Eudragit® L 100), or a chemically equivalent polymer from a different source. A concentration of the aforementioned polymer optionally ranges from 5 to 60 weight percents of the total weight of the carrier. In exemplary embodiments, a concentration of the polymer is in a range of from about 10 to about 40 weight percents of the carrier. In some embodiments, the concentration is in a range of from 15 to 25 weight percents of the carrier, optionally about 20 weight percents.

In exemplary embodiments, the formulation comprises huperzine A, native egg albumin, and poly(methacrylic acid-co-methyl methacrylate). In some embodiments, the formulation comprises about 0.4 weight percent huperzine A, and poly(methacrylic acid-co-methyl methacrylate) in an amount selected from the group consisting of about 10 weight percents, about 20 weight percents, about 30 weight percents and about 40 weight percents of the total weight of the formulation, with the balance being native egg albumin.

According to optional embodiments, the carrier comprises an additional component (i.e., a component other than huperzine A and native albumin) which is not a polymer as described herein. Such an additional component may optionally be water-soluble and/or lipophilic.

Examples of optional non-polymeric components include small, water-soluble molecules such as a saccharide (e.g., a monosaccharide, a disaccharide), and/or lipophilic components such as a fatty substance.

In some embodiments, the carrier comprises a fatty substance.

Suitable fatty substances include, but are not limited to, a fatty alcohol (i.e, an alcohol derivative of a fatty acid), a fatty acid, and a fatty acid ester (e.g., a wax, a triglyceride).

Examples of such fatty substances include, without limitation, lipids (e.g., triglycerides), hydrolysis products of lipids (e.g., glycerol palmitostearate), beeswax, and stearyl alcohol.

In some embodiments, the carrier comprises, in addition to native albumin, a monosaccharide or disaccharide. Optionally, the carrier consists essentially of albumin and a mono- and/or disaccharide.

As exemplified herein, such carriers are generally characterized by an increased huperzine A release rate, as compared to carriers comprising albumin alone.

The disaccharide is optionally lactose. A concentration of lactose optionally ranges from 10 to 70 weight percents of the total weight of the carrier, and optionally from 15 to 60 weight percents.

In some embodiments, a concentration of lactose optionally ranges from 15 to 25 weight percents of the total weight of the carrier. In exemplary embodiments, the concentration is about 20 weight percents.

In alternative embodiments, a concentration of lactose optionally ranges from 40 to 60 weight percents of the total weight of the carrier. In exemplary embodiments, the concentration is about 50 weight percents.

According to some exemplary embodiments, the formulation consists essentially of huperzine A and native egg albumin. Exemplary concentrations of huperzine A include 0.4 weight percent, 0.5 weight percent, and 1 weight percent of the total weight of the formulation, with the balance of the formulation being native egg albumin. According to optional embodiments, a concentration of huperzine A in any of the formulations described herein ranges from 0.1 to 10 weight percents of the total weight of the formulation. Optionally, the concentration of huperzine A ranges from 0.2 to 3 weight percents of the total weight of the formulation. In exemplary embodiments, the concentration of huperzine A ranges from about 0.4 to about 1 weight percent of the total weight of the formulation. Other huperzine A concentrations are also contemplated.

In some embodiments, the formulation may optionally comprise one or more components other than the carrier and the huperzine A. For example, the formulation may optionally comprise a capsule shell, a coating (e.g., an enteric coating), and/or a liquid, in addition to the carrier and huperzine A. Optionally, the carrier comprises at least 20 weight percents, optionally at least 50 weight percents, optionally at least 70 weight percents, optionally at least 80 weight percents, and optionally at least 90 weight percents of the total weight of the formulation.

Alternatively, the formulation essentially consists of the carrier and huperzine A.

Thus, optionally, the formulation primarily consists of native albumin, i.e., at least 50 weight percents of the formulation is native albumin. Optionally, at least 60 weight percents of the formulation is native albumin, optionally at least 70 weight percents, optionally at least 80 weight percents, optionally at least 90 weight percents, optionally at least 95 weight percents, and optionally at least 99 weight percents of the formulation is native albumin.

Additional compounds which may optionally be included in the formulation, in the carrier and/or outside of the carrier, include, without limitation, flavorings, colorants, emulsifiers, thickeners, disintegrants (e.g., crospovidone, crosslinked sodium carboxymethyl cellulose, sodium starch glycolate), fillers, binders, glidants (e.g., magnesium stearate, colloidal silicon dioxide, starch, talc), and/or lubricants. The additional ingredients (e.g., the aforementioned ingredients) should not result in the formulation having substantially different performance than as described herein.

The huperzine A formulation described herein can be in a form such as, but not limited to, a sachet, a pill, a caplet, a capsule, a tablet, a gel, granules, and/or a suspension of granules in water or a non-aqueous medium.

According to optional embodiments of the present invention, the formulation described herein is in the form of a sustained-release formulation unit dosage form, being for oral administration.

The term “unit dosage form”, as used herein, describes physically discrete units, each unit containing a predetermined quantity of huperzine A calculated to produce the desired therapeutic effect, in association with a carrier as described herein, and optionally with any other component of a formulation as described herein.

As used herein, the phrase “desired therapeutic effect” refers to the expected contribution of a dosage to a treatment (as defined herein), as determined by a physician, and may comprise abrogating, substantially inhibiting, slowing or reversing the progression of a condition, substantially ameliorating clinical or aesthetical symptoms of a condition, substantially preventing the appearance of clinical or aesthetical symptoms of a condition (i.e., prophylaxis), and/or raising a level of huperzine A in a body (e.g., in the blood) to a degree which may, by itself or in combination with the desired therapeutic effect of additional dosages, produce any of the aforementioned effects.

The quantity of huperzine A included in a unit dosage form depends on the condition being treated and on the desired regimen.

As indicated herein, in some embodiments, the quantity of huperzine A is a unit dosage form is such that provides a desired therapeutic effect when administered once a day.

Unit dosage forms suitable for oral administration include sachets, pills, caplets, capsules, tablets, or discrete (e.g., separately packaged) units of granules, or suspensions of granules in water or non-aqueous media.

The concentration of huperzine A in the formulation can be manipulated so as to suit a desired therapeutically effective dose and/or a desired size of a unit dosage form of the formulation.

Thus, for example, in a unit dosage form comprising from 20 to 100 ng huperzine A, the huperzine A may optionally comprise 0.1 weight percent, and optionally even less, of a unit dosage form weighing from 20 to 100 mg. In comparison, in a unit dosage form comprising at least 10 mg huperzine A, the huperzine A may optionally comprise approximately 10 weight percents of a unit dosage form weighing 100 mg or more. In exemplary embodiments, a unit dosage form may comprise from about 400 ng to about 1 mg huperzine A, the huperzine A comprising from about 0.4 to about 1 weight percent of a unit dosage form weighing from about 100 mg to about 200 mg, optionally about 100 mg.

The unit dosage form optionally comprises huperzine A in an amount that ranges from 20 μg to 50 mg, and optionally from 20 μg to 10 mg.

In some embodiments, the unit dosage form comprises at least 50 μg huperzine A, optionally at least 100 ng, optionally at least 200 ng, and optionally at least 300 μg.

In some embodiments, the unit dosage form comprises 5 mg huperzine or less, optionally 2 mg or less (e.g., from 50 μg to 2 mg), optionally 1 mg or less (e.g., from 100 μg to 1 mg), and optionally 500 μg or less (e.g., from 200 μg to 500 μg).

The amount of huperzine A may depend on the indication for which the dosage form is intended. Thus, as described in more detail elsewhere herein, a large amount of huperzine A (optionally at least 1 mg, optionally at least 2 mg, and optionally at least 5 mg) may optionally be suitable for treating a severe (e.g., life-threatening) condition, whereas a low amount of huperzine A (optionally 1 mg or less, optionally 500 μg or less) may optionally be suitable for treating a minor (e.g., non-life-threatening) condition.

According to exemplary embodiments, the formulation is in a tablet form.

As exemplified herein, the release rate can be manipulated to some extent by the size and shape of the tablet. In general, for any given width of the tablet (e.g., diameter of a cylindrical tablet), the release rate can be decreased by increasing a mass of the tablet (e.g., increasing the thickness of the tablet), whereas for any given mass of a tablet, the release rate can be decreased by decreasing a width of the tablet (e.g., decreasing a diameter of a cylindrical tablet), thereby decreasing the surface area of the tablet.

In some embodiments, a weight of the tablet is in a range of from 50 mg to 300 mg, and optionally from about 75 mg to about 200 mg. In exemplary embodiments, the weight is about 100 mg.

In some embodiments, the tablet is cylindrical and is characterized by a diameter in a range of from 4 to 10 mm, and optionally from about 5 mm to about 7 mm.

In some embodiments, the tablet is cylindrical and is characterized by a ratio of the weight-to-diameter which is in a range of from 10 to 40 mg per mm, and optionally from 12 to 30 mg per mm. In exemplary embodiments, the ratio is about 14 mg per mm.

Alternatively, the tablet is non-cylindrical but has a cross-section (perpendicular to the shortest axis of the tablet) area and/or ratio of weight-to-cross-section area which is the same as a cross-section area or ratio of weight-to-cross-section area of the above-described cylindrical tablets.

In exemplary embodiments, the tablet is cylindical, comprising a diameter of about 7 mm, and weighing about 100 mg. In some embodiments, the tablet comprises 400 ng huperzine A. In alternative embodiments, the tablet comprises about 1 mg huperzine A.

As exemplified herein, the sustained-release of the formulation described herein (including a unit dosage form described herein) may be characterized by a release profile of huperzine A (e.g., according for the time necessary for 50% of the huperzine A to be released) when the formulation is incubated in a solution which approximates physiological conditions.

In exemplary embodiments, the formulation is incubated in 0.2 M phosphate buffer at a pH of 6.8 and a temperature of 37° C., as described herein.

Thus, according to some embodiments, the formulation is characterized by a release of 50% of the huperzine A upon being incubated in the above-mentioned conditions for a time period in a range of from 1 to 10 hours.

Optionally, the time period is at least 1.5 hours, optionally at least 2 hours, optionally at least 3 hours, and optionally at least 4 hours. In some embodiments, such a sustained-release rate is obtained by including a component in the carrier which decreases the release rate (e.g., as described elsewhere herein).

Optionally, the time period upon which huperzine A release is 50% is 8 hours or less, and optionally 6 hours or less.

Optionally, the time period upon which huperzine A release is 50% is less than two hours. In some embodiments, such a sustained-release rate is obtained by using a carrier consisting essentially of albumin, or by including a component in the carrier which decreases the release rate (e.g., as described elsewhere herein) or does not substantially affect the release rate.

Without being bound by any particular theory, it is believed that the above-described release kinetics are particularly advantageous in that release is gradual enough to provide a relatively stable plasma concentrations of huperzine A over 24 hours, while also being rapid enough to allow absorption of a considerable proportion of the huperzine A in the duodenum and/or jejunum, where absorption of huperzine is more efficient than in the colon, as demonstrated herein.

Alternatively or additionally, the formulation is characterized in that it does not exhibit a delayed-release, that is, significant release of huperzine A occurs at, or shortly after, the beginning of incubation. Thus, the formulation is optionally characterized by a release of at least 10%, optionally at least 20%, and optionally at least 30%, of the huperzine A upon being incubated in the abovementioned conditions for 1 hour.

As exemplified in the Examples section, in some embodiments the release profile is biphasic, wherein the release profile characterized by an initial burst release, such that release of huperzine A at the beginning of incubation is at a relatively rapid rate, which is then followed by a second stage, wherein release occurs at a markedly slower rate. Optionally, the burst release comprises release of at least 10%, and optionally at least 20% of the huperzine A, and optionally at least 30% of the huperzine A, within 15 minutes of incubation in the abovementioned conditions.

The burst release is preferably not so large so as to deplete the huperzine A in the formulation after a short time period. Optionally, the burst release comprises release of no more than 40% of the huperzine A within 15 minutes, such that at least 60% of the huperzine A remains after 15 minutes.

In some embodiments, huperzine A is released at a higher rate in the stomach than in the intestines. Such behavior may result in a biphasic release profile comprising a relatively fast release during the time the formulation is in the stomach, followed by a slower release after the formulation has passed into the intestines.

As exemplified herein, the huperzine A release in a stomach may be characterized using simulated gastric fluid.

Thus, according to some embodiments, the formulation is characterized by a release of at least 30% of the huperzine A upon incubation for 30 minutes at 37° C. in U.S. Pharmacopeia simulated gastric fluid. Such a release characteristic will provide a moderate burst release in vivo, even in a formulation for which no burst release occurs upon incubation in phosphate buffer (e.g., as described hereinabove).

According to some embodiments, the formulation is characterized by a release of no more than 50% of the huperzine A upon incubation for 30 minutes at 37° C. in U.S. Pharmacopeia simulated gastric fluid, such that a considerable amount of the huperzine A will remain upon passage of the formulation into the intestines.

Herein, “U.S. pharmacopeia simulated gastric fluid” refers to simulated gastric fluid prepared according to U.S. Pharmacopeia 23, with pepsin. The pH of such fluid is 1.2.

As further exemplified herein, the sustained-release of the formulation described herein (including a unit dosage form described herein) may be characterized by the plasma concentration of huperzine A in a human subject when the formulation is orally administered to the subject (e.g., an average plasma concentration obtained by averaging results from a plurality of subjects). For example, the plasma concentration may be characterized as being relatively stable following administration over a time period of 24 hours.

Thus, according to some embodiments, a stability of a plasma concentration of huperzine A is such that upon oral administration of the formulation, a plasma concentration of at least 30% (optionally at least 40%, and optionally at least 50%) of the maximal plasma concentration (Cₘₐₓ) is maintained for at least 24 hours.

Alternatively or additionally, a stability of a plasma concentration of huperzine A is such that upon oral administration of the formulation (e.g., a formulation unit dosage form described herein), a plasma concentration of at least 0.75 ng/ml (optionally at least 1 ng/ml, and optionally at least 1.25 ng/ml) is maintained for at least 24 hours.

Alternatively or additionally, a stability of a plasma concentration of huperzine A is such that upon oral administration of the formulation (e.g., a formulation unit dosage form described herein), an acetylcholinesterase activity in the blood is reduced by at least 15% (optionally at least 25%, and optionally at least 40%) for at least 24 hours.

As described elsewhere herein, the formulation optionally begins to release huperzine A immediately (i.e., the release is not a delayed-release), and is optionally characterized by an initial burst release.

Hence, according to some embodiments, upon oral administration of the formulation, a maximal plasma concentration (Cₘₐₓ) is achieved relatively rapidly (e.g., due to an initial burst described herein), for example, in 2 hours or less, optionally in 1.5 hours or less, and optionally in 1 hour or less.

The stability of huperzine A levels in the blood, as described herein, may be determined by administering a single dose of the formulation (e.g., a bolus), and measuring the relevant parameter (e.g., plasma concentration of huperzine A, acetylcholinesterase activity) using methods known in the art. It is to be appreciated that a 24 hour period described hereinabove during which huperzine levels are relatively stable does not necessarily begin immediately upon administration, as there is typically a brief lag period between administration and appearance of substantial quantities of huperzine A in the blood.

The formulation described herein (including a unit dosage form described herein) is optionally identified for use in treating a medical condition treatable by huperzine A. Optionally, the formulation is for being administered once per day.

The stability of huperzine A levels in the blood following administration of a formulation described herein allows for a more effective therapy (e.g., by minimizing troughs in huperzine A levels) with less adverse side effects (e.g., by reducing peaks in huperzine A levels), as compared with what would be expected when huperzine A levels increase and decrease considerably following administration (e.g., following administration of an instant-release formulation). In addition, the stability of huperzine A levels in the blood following administration of the formulation allows for administration once per day (rather than twice or thrice per day), which often enhances patient compliance.

Hence, according to another aspect of embodiments of the invention, there is provided a method of treating a medical condition treatable by huperzine A. The method comprises orally administering a formulation described herein (e.g., one or more of a formulation unit dosage form described herein) to a subject in need thereof. Optionally, the oral administration is effected once per day.

According to another aspect of embodiments of the invention, there is provided a use of native albumin and huperzine A in the manufacture of a medicament for treating a medical condition which is treatable by huperzine A. In some embodiments, the medicament is for oral administration. Optionally, the medicament is for being administered once per day.

In any of the aspects of the invention described herein, a medical condition is considered treatable by huperzine A, when exposure to huperzine A can be reasonably expected to effect a successful treatment (and/or optimal treatment).

As used herein, the terms “treating” and “treatment” include abrogating, substantially inhibiting, slowing or reversing the progression of a condition, substantially ameliorating clinical or aesthetical symptoms of a condition or substantially preventing the appearance of clinical or aesthetical symptoms of a condition (i.e., prophylaxis). The purpose of the treatment will depend on the condition being treated, as described in more detail elsewhere herein.

As described hereinabove, huperzine A exhibits an antagonistic effect towards acetylcholinesterase and N-methyl-D-aspartate receptor.

Hence, in some embodiments, the medical condition is associated with an activity of a protein selected from the group consisting of an acetylcholinesterase and an N-methyl-D-aspartate receptor.

In some embodiments, the medical condition is a central nervous system disease or disorder, and/or a neurodegenerative disease or disorder. The disease or disorder may be a disease or disorder induced and/or enhanced by ischemia, exposure to a toxic material, trauma, head injury, or aging.

Examples of medical conditions treatable by huperzine A include, without limitation, Alzheimer's disease, memory loss (e.g., senescent forgetfulness), vascular dementia, schizophrenia, inflammation, organophosphate intoxication (e.g., by pesticides or chemical weaponry), epilepsy, ischemia, and pain.

Thus, for example, due to the effectiveness of huperzine A against inflammation and/or pain, formulations described herein may be used for a variety of applications, such as treatment of contusions, strains and/or swelling.

The exact formulation and dosage can be chosen by the individual physician in view of the patient's condition (see e.g., Fingl et al., 1975, in “The Pharmacological Basis of Therapeutics”, Ch. 1 p. 1).

The amount of a composition to be administered will, of course, be dependent on the subject being treated, the severity of the affliction, the manner of administration, the judgment of the prescribing physician, etc.

In general, administration of a large amount of huperzine A (optionally at least 1 mg, optionally at least 2 mg, and optionally at least 5 mg) may optionally be suitable for treating a severe (e.g., life-threatening) condition and/or a condition wherein an immediate therapeutic effect is desired. Examples of such treatments include treating organophosphate poisoning (e.g., during, after or shortly before exposure to the organophosphate), a severe seizure (e.g., treating a seizure which has already begun), or a severe (e.g., life-threatening) inflammation.

In general, administration of a low amount of huperzine A (optionally 1 mg or less, optionally 500 μg or less) may optionally be suitable for treating a minor (e.g., non-life-threatening) condition and/or a condition wherein an immediate effect is not necessary. Examples of such treatments include treating moderate or minor inflammation and/or pain, and regular treatment of a chronic condition (e.g., Alzheimer's disease, epilepsy, memory loss, dementia, schizophrenia).

In embodiments, wherein the treatment comprises regular administration of the formulation (as opposed to a single administration or a small number of administrations), the daily dosage of huperzine A optionally ranges from 20 μg to 50 mg, and optionally from 20 μg to 10 mg.

In some embodiments, the daily dosage comprises at least 50 μg huperzine A, optionally at least 100 ng, optionally at least 200 ng, and optionally at least 300 μg.

In some embodiments, the daily dosage comprises 5 mg huperzine or less, optionally 2 mg or less (e.g., from 50 μg to 2 mg), optionally 1 mg or less (e.g., from 100 μg to 1 mg), and optionally 500 μg or less (e.g., from 200 μg to 500 μg).

The abovementioned dosages may optionally be adjusted so as to be suitable for the body size of a particular subject.

Thus, in some embodiments, the abovementioned dosages are used for a person of average body weight (e.g., 70 kg), such that a dosage of, e.g., 1 mg, refers to a dosage of 1 mg per 70 kg body weight. Thus, a subject weighing 35 kg would receive a dosage of 0.5 mg.

In alternative embodiments, the abovementioned dosages are used without adjustment for body weight.

In some embodiments, the treatment is prophylactic. For example, the formulation may be administered to treat organophosphate intoxication before any intoxication has occurred (e.g., when there is merely a possibility of exposure to an organophosphate), to prevent a seizure (e.g., an epileptic seizure) before a seizure begins, to treat ischemic damage before induction of ischemia (e.g., before induction of ischemia during surgery), and/or to prevent memory loss (e.g., in an aging person) before any memory loss has occurred.

In some embodiments, treatment is in response to an appearance of the medical condition (rather than prophylactic).

According to another aspect of embodiments of the invention, there is provided a process of preparing a formulation as described herein (including a unit dosage form described herein), the process comprising blending huperzine A and native albumin so as to form a homogenous mixture. Optionally, the huperzine A and albumin are each in the form of a powder.

The blending may be effected using any apparatus suitable for blending.

In exemplary embodiments, the blending is effected by geometrical blending, in order to provide a more homogeneous mixture. Optionally, similar quantities of huperzine A and albumin (and/or any additional component(s) in the formulation) are blended, followed by blending of the obtained mixture with an additional quantity of albumin (and/or any additional component(s)), followed by blending of an additional quantity of albumin (and/or any additional component(s)), and so forth, until all of the components have been blended.

Optionally, additional ingredients (e.g., additional components of the carrier, as described herein) are blended with the huperzine A and albumin. When three or more components are blended, the components may be mixed simultaneously or in sequence (in any order).

In some embodiments, a compound for improving the consistency of the mixture (e.g., a glidant) is added.

The blend is optionally passed through at least one sieve in order to obtain particles of a desired size (e.g., a powder, granules).

In some embodiments, the formulation is in a tablet form, and the process further comprises compressing the homogeneous mixture (e.g., using a suitable punch and die) so as to obtain the tablet form. The tablet form may be prepared, for example, by compressing a powder or granules.

A punch and die may be used to compress the mixture. The punch and die may be operated manually or automatically.

Compression is optionally effected by applying a pressure in a range of 3 to 10 tons to the punch and die, optionally 4 to 7 tons, and optionally 4.5 to 6.5 tons. In exemplary embodiments, a pressure of about 5 tons or about 6 tons is applied.

In some embodiments, the size of the punch and die is such that a cylindrical tablet with a 7 mm diameter is formed, or a non-cylindrical tablet with an equivalent area (of the cross-section), i.e., approximately 38.49 mm²

Optionally, the pressure applied to the punch is determined according to the area of the tablet being formed. Thus, optionally, a pressure in a range of from about 3 to about 10 tons per 38.49 mm² is applied, i.e., from about 0.078 to about 0.260 tons per mm², optionally in a range of from about 0.104 to about 0.182 tons per mm², optionally in a range of from about 0.123 to about 0.169 tons per mm², and optionally in a range of from about 0.130 to about 0.156 tons per mm².

The tablet is optionally coated after compression (e.g., with an enteric coating), using any suitable technique known in the art.

Granules may optionally be prepared by wetting a powder mixture, and/or in a granulator.

In some embodiments, spheroid particles are prepared by extrusion, followed by spheronization, using techniques known in the art.

In some embodiments, the formulation is in a particulate form (e.g., granules, spheroids), either per se or combined with a diluent such as a liquid (e.g., by dispersion of the particles in the liquid).

The particles of a particulate form may optionally be individually coated (e.g., by an enteric coating) using any suitable technique known in the art.

In some embodiments, the formulation is in a capsule form. The capsule may be prepared by filling a capsule shell (e.g., a capsule shell used in the art) with a mixture of huperzine A and albumin (as described herein). The mixture may in the form of a powder, granules, spheroids, or in a larger, bulk form (optionally produced by compression, as described herein). The capsule may optionally comprise additional components (e.g., a liquid).

Techniques for formulation and administration of drugs may be found in “Remington's Pharmaceutical Sciences” Mack Publishing Co., Easton, Pa., latest edition, which is incorporated herein by reference.

According to another aspect of embodiments of the invention, there is provided a kit comprising a plurality of unit dosage forms such as are described herein. The unit dosage forms may be presented in a pack or dispenser device, such as an FDA (the U.S. Food and Drug Administration) approved kit, which may contain one or more unit dosage forms described herein. The pack may, for example, comprise metal or plastic foil, such as, but not limited to a blister pack. The unit dosage forms may be packaged within the kit separately (e.g., in different units of a blister pack) or together (e.g., combined in a single container). The kit may further comprise instructions for using the unit dosage forms for treating a medical condition (e.g., as described herein). The kit may also comprise a notice associated with the container in a form prescribed by a governmental agency regulating the manufacture, use or sale of pharmaceuticals, which notice is reflective of approval by the agency of the form of the formulations for human or veterinary administration. Such notice, for example, may be of labeling approved by the U.S. Food and Drug Administration for prescription drugs or of an approved product insert. Formulations described herein may also be prepared, placed in an appropriate container, and labeled for treatment of a medical condition treatable by huperzine A, as is detailed herein.

Thus, according to an additional embodiment of the present invention, the formulation described herein (e.g., formulation unit dosage forms described herein) of the present invention is packaged in a packaging material and identified in print, in or on the packaging material, for use in the treatment of a medical condition treatable by huperzine A, as is defined herein.

According to further embodiments of the any of the methods, uses and formulations presented herein, the huperzine A can be combined with other active ingredients which are commonly used to treat a medical condition described herein.

As used herein the term “about” refers to ±10%.

The terms “comprises”, “comprising”, “includes”, “including”, “having” and their conjugates mean “including but not limited to”.

The term “consisting of means “including and limited to”.

The terms “consisting essentially of” and “essentially consists of” mean that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

Materials:

Carboxymethyl cellulose was obtained from Sigma-Aldrich;

Egg albumin was obtained from Sigma-Aldrich;

Ethyl cellulose (N-100) was obtained from Sigma-Aldrich;

Eudragit® L 100 was obtained from Rohm Pharma Polymers (Germany);

HPMC (Methocel; hydroxypropylmethylcellulose K100LV-CR) was obtained from Colorcon (USA);

Huperzine A was obtained from Shanghai Tauto Biotech (China).

All other materials and reagents were standard items obtained from reputable commercial sources.

All solvents used were analytical grade.

Tablet Preparation:

Tablets containing polymer and huperzine A were prepared by direct compression. Powders having the indicated ingredients were blended geometrically with a mortar and pestle (homogeneity tests were performed for all mixtures). 100 mg of the mixture was weighed and placed manually into the die of an instrumented single-punch tableting machine (Perkin Elmer) to produce tablets using flat faced punches (7 mm in diameter). The tableting pressure was six tons, except where stated otherwise. Tableting pressures of 4.5 and 6.5 tons were also tested, and found to result in the same properties as when a pressure of 6 tons was used (data not shown).

Egg Albumin Denaturation:

Egg albumin was denatured by two different mechanisms: by heating and by ethanol vapor. Denaturation of the albumin was performed after preparation of the tablets. Heat denaturation was effected by placing the tablets in an autoclave for 20 minutes at a temperature of 121° C. Ethanol denaturation was effected by placing the tablets in an ethanol-filled dessicator for 2 hours, on a special surface behind the ethanol.

Evaluation of In Vitro Drug Release from Tablets:

Dissolution tests were carried out in a Caleva 7ST USP type II dissolution apparatus (G. B. Caleva Inc.). The sustained release formulation containing huperzine A was placed in a 1000 ml Caleva glass vessel containing 500 ml of release medium (0.2 M phosphate buffer, pH 6.8, except where indicated otherwise) which simulates pH conditions of the gastrointestinal tract. The vessels were closed and incubated at a temperature of 37° C.±0.5° C., with stiffing at 100 revolutions per minute. Sink conditions were maintained throughout all studies. Samples of 100 μl were withdrawn at the indicated times during the dissolution process. The dissolution tests were carried out in triplicate. The amount of huperzine A released was evaluated using an HPLC assay, using to a modification of the procedures described by Li et al. [Eur J Drug Metab Pharmacokinet 2007, 32:183-187].

In Vitro Permeability Study (Using Caco-2 Cells):

Caco-2 cells were obtained from ATCC (Manassas, Va., USA) and grown in 75 cm² flasks with approximately 0.5-1×10⁶ cells per flask, at a temperature of 37° C. in a 5% CO₂ atmosphere with a relative humidity of 95%. The culture growth medium consisted of Dulbecco's Modified Eagle Medium (DMEM) supplemented with 10% heat-inactivated fetal bovine serum (FBS), 1% nonessential amino acids, and 2 mM L-glutamine. The medium was replaced twice weekly. All medium supplements were obtained from Biological Industries (Beth-Haemek, Israel).

Cells in a passage range of 52-60 were seeded at a density of 25×10⁵ cells/cm² on untreated culture inserts of a polycarbonate membrane with 0.4 μm pores and a surface area of 1.1 cm². The culture inserts containing Caco-2 monolayers were placed in Costar® 24-Transwell plates (12 mm wells). The culture medium was changed every other day. Studies were performed 21-22 days after seeding, when the cells were fully differentiated, and the TEER (trans-epithelial electrical resistance) values were stable (300-500 ohm/cm²).

Each study was initiated by replacement of the medium on the apical and basolateral sides of the monolayer with 600 μl apical buffer and 1500 μl basolateral buffer, respectively, each buffer being warmed to 37° C. The cells were incubated for a 30 minute period at 37° C. with shaking (100 cycles per minute). After the incubation period, the buffers were removed and replaced with 1500 μl basolateral buffer on the basolateral side, and 600 μl apical buffer with test solutions (warmed to 37° C.) on the apical side. 50 μl of samples were taken from the apical side immediately at the beginning of the experiment, resulting in a 550 μl apical volume during the experiment. For the duration of the experiment, the cells were kept at 37° C. with shaking. At predetermined times (30, 60, 90, 120, 150 and 180 minutes), 200 μl samples were taken from the basolateral side and replaced with 200 μl of fresh basolateral buffer to maintain a constant volume.

The permeability coefficient (Papp) for each compound was calculated from the linear plot of drug accumulated versus time, using the following equation:

Papp=dQ/dt/(Cₒ·A)

where dQ/dt is the steady state rate of appearance of the drug on the receiver side, C₀ is the initial concentration of the drug on the donor side, and A is the surface area, 1.1 cm².

Ex-Vivo Animal Permeability Study:

Permeability experiments were performed in a modified Us sing chamber system (Physiological Instruments Inc., San Diego, Calif.). Male Sabra rats, weighing 275-325 grams, were used. Following a midline incision, the intestine was removed and placed in ice-cold Ringer bicarbonate buffer (6.54 grams NaCl, 0.37 gram KCl, 0.18 gram CaCl₂.2H₂O, 0.24 gram MgCl₂.6H₂O, 2.1 grams NaHCO₃, 0.23 gram Na₂HPO₄, and 0.05 gram NaH₂PO₄ in 1000 ml). All buffer solutions were freshly prepared and equilibrated to a pH of 7.4. The duodenal portion of the small intestine (3-5 cm distal to the pylorus) was used. Peyer patches could be easily identified visually, and sections containing them were not used. The individual segments were obtained, and underlying muscularis was removed from the serosal side of the tissue before mounting. The exposed tissue surface area was 1.12 cm² and the fluid volume in each half-cell was 3 ml. The system was preheated to a temperature of 37° C. Modified Ringer buffers were added to the serosal and the mucosal sides; mucosal modified Ringer buffer contained 10 mM mannitol, and serosal modified Ringer buffer contained 8 mM D-glucose and 2 mM mannitol. Tissue oxygenation and mixing of the solution were achieved by bubbling with 95% O₂-5% CO₂. The system was equilibrated for 20 minutes. The permeability experiments continued for 150 minutes, during which samples were withdrawn at predetermined time periods. The sampled volume was replaced by blank (non-compound containing) buffer to maintain sink conditions. The integrity of epithelial tissue was monitored by measuring the trans-epithelial electrical resistance (TEER) throughout the experiment. Generally, TEER values were 70-130 Ω·cm² and remained steady throughout the experiment.

The amount of permeated huperzine A was determined using a HPLC assay. Cumulative corrections were made for the previously removed samples.

The permeability coefficient (Papp) for each compound was calculated from the linear plot of drug accumulated vs. time, using the equation described hereinabove, wherein the exposed tissue area (A) is 0.5 cm².

Pharmacokinetic Studies in Rats:

All surgical and experimental procedures were reviewed and approved by the Animal Experimentation Ethics Committee of the Hebrew University Hadassah Medical School, Jerusalem. Male Wistar rats (Harlan, Israel), 290-350 grams in weight, were used for all surgical procedures.

Animals were anesthetized for the period of surgery by intra-peritoneal injection of 1 ml/kg of ketamine (9%)-xylazine (1%) solution. An indwelling cannula was placed in the right jugular vein of each animal for systemic blood sampling, as described in Jiang et al. [Curr Med Chem 2003, 10:2231-2252]. The cannula was tunneled beneath the skin and exteriorized at the dorsal part of the neck. After completion of the surgical procedure, the animals were transferred to metabolic cages to recover overnight (12-18 hours). During this recovery period and throughout the experiment, food, but not water, was deprived. Animals were randomly assigned to the different experimental groups.

In order to study the pharmacokinetic parameters of huperzine A, identical doses (0.5 mg/kg) of huperzine A dissolved in water were administered to 6 animals by oral gavage needle and to 6 animals by intravenous infusion through the jugular vein cannula followed by injection of 0.2 ml of heparinized saline (50 IU/ml) to ensure the delivery of the whole dose. Systemic blood samples (400 μl) were taken 5 minutes prior to dosing, and at various time periods post-dose, as indicated. To prevent dehydration, equal volumes of physiological solution were introduced to the rats following each withdrawal of a blood sample.

Another group (4 animals) received huperzine A as an intra-peritoneal injection. The doses, the vehicle and the volume used in the intra-peritoneal administration were similar to those used in the oral administration experiments.

Absorption of huperzine A from the colon was studied using an additional experimental group (7 animals). A cannula was placed to the cecum, tunneled beneath the skin and exteriorized at the dorsal part of the neck. Following overnight recovery, the animals were dosed through the colonic cannula and the pharmacokinetic experiment was conducted. The doses, the vehicle and the volume used in the colonic administration were similar to those used in the oral administration experiments. The drug was infused over the course of 90 minutes.

Absorption of huperzine A from the duodenum was studied using another experimental group (5 animals). A cannula was placed to the duodenum, tunneled beneath the skin and exteriorized at the dorsal part of the neck. Following overnight recovery, the animals were dosed through the duodenal cannula and the pharmacokinetic experiment was conducted. The doses, the vehicle and the volume used in the duodenal administration were similar to those used for the other administration routes. Huperzine A was infused to the duodenum over the course of 90 minutes.

Huperzine A concentrations in plasma samples were determined by an HPLC assay. The area under plasma concentrations versus time curves (AUC) for huperzine A in individual rats were analyzed using WinNonlin® Professional software version 5.0.1, according to the non-compartmental analysis model, as were other pharmacokinetic parameters (elimination constant (10; elimination half-life (T_{1/2}); peak concentration (Cₘₐₓ); and time to reach peak concentration (Tₘₐₓ)). The absolute bioavailability (F) of huperzine A was calculated from the ratio of the AUCs normalized by dose after oral and intravenous administration.

HPLC Assay for Huperzine A:

All tested compounds were analyzed using a Waters 2695 Separation Module HPLC system with a Waters 2475 Fluorescence Detector and a Waters 2996 Photodiode Array Detector (Waters Corporation). The analytical procedure for Huperzine A in plasma samples was based on a modification of the procedures described in Li et al. [Eur J Drug Metab Pharmacokinet 2007, 32:183-187].

150 μl samples were mixed with 150 μl of NaOH (1 M) solution and 15 μl of an internal standard (antipyrine). Materials were extracted with 4 ml of ethyl acetate, evaporated to dryness, and reconstituted with 120 μl of water.

The volume of injection was 40 μl. The separation was achieved by Hypersil GOLD C8 column (5 μm, 4.6×150 mm) at 40° C. The mobile phase consisted of methanol:water (45:55 v/v), adjusted to a pH of 10 with triethylamine. The flow was set to 1 ml/minute. Huperzine A was detected with an excitation wavelength of 310 nm and an emission wavelength of 370 nm, and retention time was 4.7 minutes. The calibration curves were linear between 2 and 10,000 ng/ml.

### Example 1

### Intestinal Permeability Towards Huperzine A

Intestinal permeability towards huperzine A was evaluated using an in vitro model with Caco-2 epithelial colorectal cell culture, and using an ex-vivo animal intestine model (Ussing chamber model), as described in the Materials and Methods section.

Antipyrine was used as a marker for transcellular permeability, whereas mannitol was used as an example of a compound which undergoes paracellular transport.

As shown in FIG. 1, the permeability coefficient (Papp) of huperzine A was lower than that of antipyrine, and higher than that of mannitol, is determined using a Caco-2 cell culture.

As shown in FIG. 2, the permeability towards huperzine A decreased along the gastrointestinal tract, but was absorbed at all parts of the gastrointestinal tract. The permeability in the colon was lower than in the jejunum and duodenum, but was still significant.

As further shown in FIG. 2, the permeability coefficient of huperzine A was lower than that of antipyrine, in agreement with the results obtained with Caco-2 cells. Similarly, the permeability coefficient of huperzine A was higher than that of mannitol (not shown).

These results indicate that huperzine A crosses the intestinal border by passive diffusion.

### Example 2

### Pharmacokinetics of Huperzine A

The pharmacokinetics of huperzine A was evaluated by measuring plasma concentrations of huperzine A in rats following administration of 0.5 mg/kg huperzine A via oral (p.o.), intravenous (i.v.), intraperitoneal (i.p.) and colonic and duodenal infusion routes, as described in the Materials and Methods section.

As shown in FIGS. 3 and 4, huperzine A exhibited rapid absorption, with maximal plasma levels (Cₘₐₓ) occurring at about 25-30 minutes after oral administration, as well as rapid elimination, with an elimination half-life (T_{1/2}) of approximately 100 minutes. As further shown therein, the oral bioavailability of huperzine A (F) was about 50%, as determined by comparison with huperzine A levels following intravenous (FIG. 3) and intraperitoneal (FIG. 4) administration.

As shown in FIG. 5, bioavailability following duodenal infusion was at least as high as the bioavailability following oral bolus, whereas bioavailability following colonic infusion was significantly lower than the bioavailability following oral bolus. This indicates that huperzine A may be absorbed somewhat less effectively in the colon.

### Example 3

### Effect of Egg Albumin-Based Formulations on Huperzine A Pharmacokinetics Huperzine A sustained release tablets were prepared based on native egg albumin, and the pharmacokinetic profile of the huperzine A was then tested in vivo in rats.

Huperzine A tablets were prepared with a matrix comprising a combination of egg albumin and lactose or egg albumin alone. The tablets were 2 mm in diameter, and 5 mg in weight, and the administered dose of huperzine A was 0.5 mg/kg body weight. The composition of the tablets (by weight) was 3% huperzine A and 97% native egg albumin, or 3% huperzine A, 67% native egg albumin and 30% lactose. The results were compared with the pharmacokinetic profile obtained following oral administration of huperzine A as described in Example 2.

As shown in FIG. 6, the native egg albumin-based formulation exhibited sustained release of huperzine A. Formulations containing native egg albumin alone as a matrix exhibited a more sustained release than did formulations comprising native egg albumin and lactose.

These results indicate that formulations containing native egg albumin provide a sustained release of huperzine A, resulting in a longer lasting therapeutically effective plasma concentration of huperzine A.

### Example 4

### Effect of Heat Treatment on Huperzine A

The stability of huperzine A under heat treatment was tested both alone in solution and by heat-treatment of huperzine A-containing tablets.

0.5 ml of a 1 μg/ml aqueous solution of huperzine A was placed in glass tubes and heated in an oven for 30, 60 or 90 minutes. The samples were then reconstituted with double-distilled water and the amount of remaining huperzine A was determined by HPLC, as described hereinabove.

In addition, tablets were prepared from huperzine A (1%) and native egg albumin (99%), and the egg albumin was then denatured by heat treatment or ethanol treatment, as described in the Materials and Methods section. Drug release from the tablets was then tested in a dissolution apparatus at a pH of 6.8, as described in the Materials and Methods section.

As shown in FIG. 7, huperzine A alone exhibits sensitivity to heat treatment, with degradation of the huperzine A being observed at temperatures of 150° C. or more.

In addition, huperzine A was even more sensitive to heat treatment when in a tablet. Following heat treatment of a huperzine A tablet at 121° C. (as described hereinabove), only 86% of the huperzine A was recovered following complete dissolution of the tablet, indicating that 14% of the drug was destroyed.

As shown in FIG. 8, heat denaturation of egg albumin in tablets decreased the release of huperzine A in comparison with tablets containing native egg albumin, whereas ethanol denaturation had relatively little effect on release of huperzine A.

In view of the sensitivity of huperzine A to heat treatment, sustained release tablets were prepared using native egg albumin, without undergoing heat treatment.

### Example 5

### Effect of Native Egg Albumin-Based Formulations on Huperzine A Release Profiles

In order to further characterize the effect of the formulation composition on the release profile of huperzine A, tablets were prepared with a variety of matrix compositions, and drug release was tested in a dissolution apparatus as described in the Materials and Methods section.

In addition to native egg albumin, some of the tested tablet matrices contained lactose, Eudragit® L 100 (a polymer with a pH-sensitive solubility) hydroxypropylmethylcellulose (HPMC K100LV-CR), ethyl cellulose (N-100), poly(ethylene oxide) (Polyox WSR-303), hydroxypropyl cellulose (Klucel® HF), or carboxymethyl cellulose.

The compositions of the tablets, which weighed 100 mg (except for one formulation in which tablets weighed 200 mg) and contained 1 mg of huperzine A, are presented in Table 1 below.

The effect of tablet size and shape on huperzine A release was assessed by comparing the dissolution of 100 mg tablets (Formulation 1) with diameters of 5 mm and 7 mm and of a 200 mg tablet (Formulation 2) with a 7 mm diameter.

As shown in FIG. 9, the 200 mg tablet released huperzine A more gradually than did the 100 mg tablets. As further shown therein, the 7 mm tablet released huperzine A more rapidly than did the 5 mm tablet.

These results indicate that the rate at which huperzine A is released from tablets is correlated to the diameter of the tablet (for any given tablet weight) and inversely correlated to the tablet weight (for any given tablet diameter).

As further shown in FIG. 9, the release of huperzine A followed a biphasic profile, wherein approximately the first 20% of the drug was released in a “burst” with more rapid kinetics than the later release.

The effect of lactose on huperzine A release was assessed using tablets containing about 20% (Formulation 4) or 50% lactose (Formulation 3), or no lactose (Formulation 1).

As shown in FIG. 10, inclusion of lactose in the tablet enhanced the rate of release of huperzine A from the tablet, in a concentration-dependent manner.

These results indicate that lactose enhances the rate of release, presumably by increasing the porosity of the tablet.

TABLE 1
Exemplary tablet compositions

| | | Egg | | | | | | | |
|---|---|---|---|---|---|---|---|---|---|
| | | albumin | | Eudragit | | | | | |
| | Huperzine A | (native) | Lactose | L 100 | HPMC | EC | CMC | PEO | HPC |
| Formulation | (mg) | (mg) | (mg) | (mg) | (mg) | (mg) | (mg) | (mg) | (mg) |
| 1 | 1 | 99 | | | | | | | |
| 2 | 1 | 199 | | | | | | | |
| 3 | 1 | 49.5 | 49.5 | | | | | | |
| 4 | 1 | 79.5 | 19.5 | | | | | | |
| 5 | 1 | 59.5 | | | 39.5 | | | | |
| 6 | 1 | 79.5 | | | 19.5 | | | | |
| 7 | 1 | 59.5 | | | | 39.5 | | | |
| 8 | 0.4 | 99.6 | | | | | | | |
| 9 | 0.4 | 69.6 | | | | | 30 | | |
| 10 | 0.4 | 89.6 | | 10 | | | | | |
| 11 | 0.4 | 79.6 | | 20 | | | | | |
| 12 | 0.4 | 69.6 | | 30 | | | | | |
| 13 | 0.4 | 59.6 | | 40 | | | | | |
| 14 | 0.4 | 69.6 | | | 30 | | | | |
| 15 | 0.4 | 69.6 | | | | | | 30 | |
| 16 | 0.4 | 69.6 | | | | | | | 30 |

HPMC—hydroxypropylmethylcellulose
EC—ethyl cellulose
CMC—carboxymethyl cellulose
PEO—poly(ethylene oxide)
HPC—hydroxypropyl cellulose

The effect of various polymers on the release rate of huperzine A was also determined.

As shown in FIG. 11, tablets containing native egg albumin with the hydrophilic polymer hydroxypropylmethylcellulose (HPMC) (Formulation 6) exhibited a slower rate of release than did tablets containing native egg albumin alone (Formulation 1).

As shown in FIG. 12, tablets containing native egg albumin with the hydrophobic polymer ethyl cellulose (Formulation 7) exhibited a slower rate of release than did tablets containing native egg albumin alone (Formulation 1), and a considerably faster rate of release than did tablets containing ethyl cellulose without native egg albumin.

As shown in FIG. 13, tablets containing native egg albumin with 30% carboxymethyl cellulose (Formulation 9) exhibited an approximately zero-order sustained release of huperzine A.

As shown in FIG. 14, tablets containing native egg albumin with Eudragit® L 100 at concentrations of 10% (Formulation 10), 20% (Formulation 11), 30% (Formulation 12) and 40% (Formulation 13) exhibited a slower rate of release than did tablets containing native egg albumin alone (Formulation 8). The degree to which Eudragit® L 100 slowed the release of huperzine A was proportional to the concentration of Eudragit® L 100.

As shown in FIG. 15, tablets containing native egg albumin with 30% hydroxypropylmethylcellulose (Formulation 14) or poly(ethylene oxide) (Formulation 15) exhibited a slower rate of release than did tablets containing native egg albumin alone (Formulation 8).

As shown in FIG. 16, tablets containing native egg albumin with 30% hydroxypropyl cellulose (Formulation 16) exhibited a slower rate of release than did tablets containing native egg albumin alone (Formulation 8).

The above results indicate that formulations of huperzine A based on a native egg albumin-containing matrix exhibit sustained release of huperzine A, and that the sustained release profile can be controlled by the addition of additional ingredients, such as hydrophilic and/or hydrophobic polymers.

### Example 6

### Effect of pH on Huperzine A Release Profiles

In order to further characterize the effect of the environment of the gastrointestinal tract on huperzine A release, native egg albumin-based huperzine A tablets described hereinabove (Formulation 8) were exposed to simulated physiological fluids. The dissolution of the tablets was tested as described hereinabove, except that the tablets were first incubated in simulated gastric fluid (pH 1.2, prepared according to USP 23, with pepsin) for 90 minutes, and then transferred to simulated intestinal fluid (pH 7.5, prepared according to USP 23, with pancreatin) until 72 hours after initiation of the dissolution test. The results of the dissolution test in simulated physiological fluids were compared with the results of a dissolution test in phosphate buffer with a pH of 6.8.

As shown in FIG. 17, huperzine A was released at a more rapid rate in simulated physiological fluids than in phosphate buffer (pH 6.8), due to the relatively rapid release which occurred in simulated gastric fluid (the first 90 minutes of the incubation in simulated physiological fluids). As further shown therein, a relatively large burst release occurred in simulated gastric fluid, with approximately 40% of the huperzine A being released within 30 minutes. In phosphate buffer (ph 6.8) a smaller initial burst release was observed.

These results indicate that under physiological conditions, a substantial portion of huperzine A is released from exemplary formulations in an initial burst, with the remainder of the huperzine A being released gradually in a sustained manner.

### Example 7

### Pharmacokinetics of Sustained Release Huperzine A Formulations in Humans

Huperzine A sustained release formulations were prepared based on native egg albumin, and the pharmacokinetic profile of the huperzine A was evaluated in healthy volunteers. In addition, the release profile was determined in vitro by a dissolution test as described hereinabove.

One formulation consisted of 0.4% huperzine A and 99.6% native egg albumin, and is referred to as “CR-1” or Formulation 8.

A second formulation consisted of 0.4% huperzine, 79.6% native egg albumin, and 20% Eudragit® L 100, and is referred to as “CR-2” or Formulation 11.

The ingredients of each formulation were mixed with a mortar and pestle and cylindrical tablets were prepared by direct compression of the blends, using a laboratory press (Carver Laboratory Equipment) fitted with a 7 mm flat-faced punch and die set, while applying a pressure of 5.5 tons. The sustained release tablets each weighed 100 mg.

For comparison, an instant release formulation was prepared, which consisted of 0.2% huperzine A and 99.8% lactose. The ingredients were mixed with a mortar and pestle, and 200 mg of the blend was inserted into water-soluble gelatin capsules.

As shown in FIG. 18, The sustained release formulations released huperzine A at a considerably slower rate than did the instant release formulation, with the CR-2 formulation (which contained a matrix of native egg albumin and Eudragit® L 100) providing a more sustained release than did the CR-1 formulation (which contained native egg albumin alone in the matrix).

These results are consistent with those of similar experiments described hereinabove.

A cross-over open label three-phase comparative study was then performed in eight healthy male volunteers. Each volunteer received an immediate release huperzine A encapsulated formulation and the two native egg albumin-based sustained release formulations. Each formulation comprised 0.4 mg huperzine A.

Volunteers fasted ten hours before administration of the formulation and were on a xanthine-free diet during the 24 hours pre-dose and during the study. The formulation was administered along with 400 ml of water (at 8:00 AM). Meals were served four hours (at 12:00) and eight hours (at 16:00) after administration. Volunteers remained in the unit under the supervision of the medical team until 20:00, and were required to return at 8:00 AM, 14:00 and 20:00 the next day for completion of the study.

To determine Huperzine A concentrations and to calculate the pharmacokinetic parameters, 13 to 15 blood samples were drawn (12 ml each) via a catheter during each phase of the study. Blood samples were centrifuged within 30 minutes following blood collection for 7 minutes at 4° C. (4000 rotations per minute), and the plasma was separated and divided into two aliquots of about 3 ml each in a tightly sealed plastic tube. The labeled samples were frozen at −20° C. in a vertical position and stored at this temperature pending analysis.

Huperzine A and antipyrine (internal standard) were isolated from human plasma by extraction with ethyl acetate, chromatographed on a Hypersil GOLD C8 column (5 μm, 4.6×150 mm) at 40° C. with a mobile phase consisting of aqueous formic acid (0.05%):methanol (55:45 v/v) and detected using a tandem mass spectrometer (Thermo Scientific) with an electro-spray ionization interface. The lower limit of quantification was 0.05 ng/ml, and the assay exhibited linearity in a range of 0.1-10 ng/ml.

Pharmacokinetic data from the two formulations were compared using student T-test (two-tail, paired).

As shown in FIG. 19, the CR-1 sustained release formulation resulted in a lower peak plasma concentration of huperzine A, and in a more stable plasma concentration, as compared to the instant release formulation. 24 hours after administration of the CR-1 formulation, the plasma concentration of huperzine A was about 50% of the peak plasma concentration (Cₘₐₓ), whereas 24 hours after administration of the instant release formulation, the plasma concentration was only about 20% of the peak plasma concentration. The difference between the peak plasma concentrations provided by the two formulations was statistically significant (p=0.004).

These results indicate that exemplary formulations described herein are effective for providing sustained release of huperzine A in humans, as well as relatively stable plasma concentrations for at least 24 hours.

As further shown in FIG. 19, peak plasma concentration (Cₘₐₓ) following administration of the sustained release formulation occurred at about the same time as the peak plasma concentration following administration of the instant release formulation.

Thus, these results further indicate that the sustained release of huperzine A is accompanied by rapid absorption of the drug into the bloodstream.

Additional pharmacokinetic parameters are determined essentially as described hereinabove.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A sustained-release formulation comprising huperzine A and a carrier, said carrier comprising native albumin, the formulation being for oral administration.

2. The formulation of claim 1, wherein said carrier is a solid carrier.

3. The formulation of claim 1, wherein said albumin is egg albumin.

4. The formulation of claim 1, wherein at least 50 weight percents of the carrier is said native albumin.

5. The formulation of claim 1, wherein a concentration of huperzine A ranges from 0.1 to 10 weight percents of the total weight of the formulation.

6. The formulation of claim 1, wherein said carrier further comprises a polymer.

7. (canceled)

8. The formulation of claim 6, wherein said polymer is selected from the group consisting of ethyl cellulose, methyl cellulose, hydroxypropyl cellulose, hydroxypropylmethylcellulose, hydroxyethyl cellulose, hydroxyethylmethylcellulose, carboxymethyl cellulose, poly(methacrylic acid-co-methyl methacrylate), poly(methacrylic acid-co-ethyl acrylate), poly(ethylene oxide), a poloxamer, a polyacrylamide, a polysaccharide, and a protein.

9-10. (canceled)

11. The formulation of claim 1, wherein said carrier further comprises an additional component selected from the group consisting of a saccharide, and a fatty substance.

12-15. (canceled)

16. The formulation of claim 1, comprising huperzine A in an amount selected from the group consisting of 0.4 weight percent, 0.5 weight percent, and 1 weight percent of the total weight of the formulation, with the balance being native egg albumin.

17-18. (canceled)

19. The formulation of claim 1, comprising 0.4 weight percent huperzine A, 69.6 weight percents native egg albumin, and 30 weight percents of a polymer selected from the group consisting of hydroxypropylmethylcellulose, carboxymethyl cellulose, poly(methacrylic acid-co-methyl methacrylate), poly(ethylene oxide) and hydroxypropyl cellulose.

20. (canceled)

21. The formulation of claim 1, comprising 1 weight percent huperzine A, 59.5 weight percents native egg albumin, and 39.5 weight percents ethyl cellulose.

22-24. (canceled)

23. The formulation of claim 1, comprising 0.4 weight percent huperzine A, and poly(methacrylic acid-co-methyl methacrylate) in an amount selected from the group consisting of 10 weight percents, 20 weight percents, 30 weight percents and 40 weight percents, with the balance being native egg albumin.

26-29. (canceled)

30. The formulation of claim 1, being characterized by a release of 50% of said huperzine A upon incubation in 0.2 M phosphate buffer at a pH of 6.8 and a temperature of 37° C. for a time period in a range of from 1 to 10 hours.

31. The formulation of claim 1, being characterized by a release of from 10% to 40% of said huperzine A upon incubation in 0.2 M phosphate buffer at a pH of 6.8 and a temperature of 37° C. for 15 minutes.

32. The formulation of claim 1, being characterized by a release of from 30% to 50% of said huperzine A upon incubation for 30 minutes at 37° C. in U.S. Pharmacopeia simulated gastric fluid.

33. The formulation of claim 1, being characterized by an ability, upon oral administration of the formulation to a human subject, to maintain a plasma concentration of huperzine A which is at least 30% of the maximal plasma concentration, for at least 24 hours.

34. The formulation of claim 1, being in a unit dosage form.

35. The formulation of claim 34, being characterized by an ability, upon oral administration of the unit dosage form to a human subject, to maintain a plasma concentration of at least 0.75 ng/ml huperzine A for at least 24 hours.

36-37. (canceled)

38. The formulation of claim 1, identified for use in treating a medical condition treatable by huperzine A.

39-40. (canceled)

41. A kit comprising a plurality of the unit dosage form as described in claim 34, and instructions for using said unit dosage form for treating a medical condition treatable by huperzine A.

42. A method of treating a medical condition treatable by huperzine A, the method comprising orally administering the formulation of claim 1 to a subject in need thereof, thereby treating the medical condition.

43. The method of claim 42, wherein said administering is effected once per day.

44-46. (canceled)

47. The formulation of claim 1, wherein said medical condition is associated with an activity of a protein selected from the group consisting of an acetylcholine esterase and an N-methyl-D-aspartate receptor.

48. The formulation of claim 1, wherein said medical condition is selected from the group consisting of Alzheimer's disease, memory loss, vascular dementia, schizophrenia, inflammation, organophosphate intoxication, epilepsy, ischemia, and pain.

49. A process of preparing the formulation of claim 1, the process comprising blending huperzine A and native albumin so as to form a homogeneous mixture.

50. (canceled)
